(19) United States
(12) Patent Application Publication
(10) Pub. No.: US 2005/0112066 A1
(43) Pub. Date: May 26, 2005

(54) Complexes of cyclic polyaza chelators with cations of alkaline earth metals for enhanced biological activity

(75) Inventor: Harry S. Winchell, Lafayette, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CONCAT LP, a California Limited Partnership, Concord, CA (US)

(21) Appl. No.: 10/723,439

(22) Filed: Nov. 26, 2003

(51) Int. Cl. A61K 49/00; A61K 31/555

(52) U.S. Cl. 424/9.363; 534/15; 514/184

(57) ABSTRACT

Cyclic polyaza chelators that possess high affinity and specificity for first transition series metal cations exhibit an unanticipated improvement in biological activity when administered as complexes with cations of the alkaline earth metals, Ca(II) and Mg(II), most notably Ca(II). By virtue of this improvement, these complexes are particularly effective in the treatment of pathological conditions, including ischemia and ischemia-reperfusion injury.

### BACKGROUND OF THE INVENTION

1. Field of the Invention

This invention resides in the field of chelating agents and complexes thereof, and in the biological and medical uses of such agents. This invention also resides in the field of treatment of diseases characterized by ischemia and ischemia-reperfusion injury. All literature and patent citations in this specification are hereby incorporated herein by reference.

2. Description of the Prior Art

Cations of first transition metals catalyze, or are active components in, a wide variety of biochemical reactions. A supply of such cations is required, for example, for cell and viral replication. Chelation of such cations can alter their bioavailability. Consequently, chelators of first transition series elements have the potential to influence a wide variety of biochemical reactions in non-replicating as well as replicating cells and viruses and to inhibit such replication.

In applying this potential to physiological conditions, chelators of first transition series metals have been demonstrated to possess antimicrobial and antineoplastic properties, to mitigate ischemia and ischemia-reperfusion injury, to inhibit viral replication, to inhibit the action of metalloenzymes, to remove excess iron and copper from the body, and other therapeutic and preventive physiological effects.

The chemical form of chelators can be altered in a manner that affects their specificity in achieving biological effects. Chelators that are highly hydrophilic and do not pass plasma membranes, for example, demonstrate specificity toward biological targets located in the extracellular fluid space or targets that are affected by metal cation concentrations in the extracellular fluid space.

Steric constraints imposed on the chemical structure of chelators can result in optimization of the specificity and affinity of the chelators for specific metal cations. A chelator's chemical structure and the nature of the chelated cation can also affect the ability of the chelator to gain access to in vivo sites where specific targeted metal cations are resident, as well as the ability of the chelator to form a complex with a metal cation and thereby affect the kinetics of translocation of the metal from such sites. The chelator can also be designed to bind to specific sites surrounding a metal contained in a metalloenzyme in a manner that optimizes the positioning of unbonded electron pairs in the chelator in the coordination sphere of the active metal cation, thereby inhibiting the enzymatic action of the metalloenzyme.

Steric restraints can be imposed on a chelator by imposing a relatively rigid conformational structure to the spatial orientation of the unbonded electron pairs of such atoms as oxygen, nitrogen, sulfur, and the like, of the chelator intended to occupy the coordination sphere of the complexed cation thereby limiting the size and nature of metal cations that can be optimally complexed by the chelator. Such conformational rigidity can be achieved by incorporating closed ring structures into the structure of the chelator.

Cyclic polyaza chelators are known to offer specificity and affinity for selected metal cations. It is also known however that the formation of complexes of these chelators with metal cations occurs at a slow rate relative to complexes of linear polyaza chelators with the same cations. For example, the chelator N,N′,N″-tris(dihydroxyphosphoryl methyl)-1,4,7-triazacyclononane has high affinity and specificity for cations of first transition series elements but may take many months to achieve equilibrium with the cations at room temperature. Unless they are bound in vivo, chelators that are highly aquaphilic are typically excreted rapidly from the body in the urine. Likewise, chelators that are highly lipophilic are typically excreted rapidly in bile. In both cases, the limited time that such chelators are resident in the body may be insufficient to allow the chelators to reach thermodynamic equilibrium in forming the desired complexes. For these chelators, therefore, the kinetics of complex formation may be the principal factor in determining the biological efficacy of the chelator.

As disclosed in the prior art, notably U.S. Pat. No. 5,874,573, issued Feb. 23, 1999, chelators whose therapeutic value is derived from their complexation in the body with transition metal cations that are present in the body can be administered as complexes with a different cationic component such as an alkali or alkaline earth metal cation. This is done with the expectation that the cationic component of the administered complex will be replaced in the body with the transition metal cation. Taken in isolation from other parameters, the more avidly the cationic component of the chelator is bound by the chelator at the time of its administration, the slower it should dissociate from the complex to liberate the chelator and the slower the kinetics of binding of the chelator to the targeted cation. One would therefore expect that the biological activity of chelators whose activity depends on in vivo binding of first transition series elements would decrease accordingly. Accordingly, other parameters being equal, the kinetics of dissociation of complexes between cyclic polyaza chelators and monocationic alkali metals (such as sodium, for example) to liberate free chelator should be faster, and the biological activity of the chelators greater, than when the cationic component of the administered complex is a more tightly bound dicationic alkaline earth metal cation such as calcium or magnesium.

### SUMMARY OF THE INVENTION

Contrary to expectation, it has now been discovered that the biological activity of cyclic polyaza chelators with high affinity and specificity for first transition series cations is actually enhanced, rather than reduced, by administering the chelators in the form of complexes with cations of alkaline earth metals. It has further been discovered, even more contrary to expectation, that cations of alkaline earth metals (which are dicationic), particularly calcium and magnesium, provide a significantly greater enhancement to the biological activity of the chelators than do alkali metals (which are monocationic) such as sodium. This enhancement is manifest in a variety of circumstances, including diseases against which the chelators function as therapeutic agents. Included among these conditions and diseases are ischemia and ischemia-reperfusion injury.

These and other features, aspects, embodiments, and applications of this invention will be more fully understood from the discussion that follows.

### DETAILED DESCRIPTION OF THE INVENTION AND PREFERRED EMBODIMENTS

A preferred class of cyclic polyaza chelators for use in the practice of the present invention is defined by Formula I:

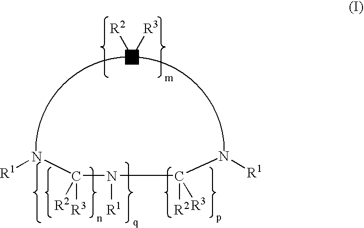

In Formula I, the indices m, n, and p are either 2 or 3, and are either all the same or one or more are 2 and the remainder(s) 3, and q is 1 or 2. All R¹'s on any single molecule defined by Formula I may be the same or one or more may be different from the other(s), the R²'s on any single molecule may likewise be the same or different, and the R³'s on any single molecule may likewise be the same or different. Each of these symbols (R¹, R², and R³) represents H, alkyl, alkenyl, aryl, arylalkyl, alkoxy, alkylthio, alkenoxy, alkenylthio, aryloxy, arylthio, alkyl interrupted by one or more oxa (—O—), alkenyl interrupted by one or more oxa (—O—), alkyl interrupted by one or more thia (—S—), alkenyl interrupted by one or more thia (—S—), aryloxyalkyl, alkoxyaryl, aminoalkyl, aminoalkenyl, aminoaryl, aminoarylalkyl, hydroxyalkyl, hydroxyalkenyl, hydroxyaryl, or hydroxyarylalkyl, provided only that these groups that do not interfere with complexation and that they are not combined in a manner that results in a chemically unstable configuration. The alkyl, alkenyl and aryl groups, or portions of groups, in the foregoing list can also be substituted with one or more halogen atoms. As further alternatives, R¹, R² and R³ can be combined to form a ring structure.

In addition to the radicals listed in the preceding paragraph, R¹ is further defined to include a radical of Formula II:

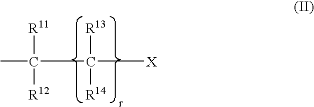

In Formula II, r is either zero or 1, and all R¹¹ 's on any single molecule may be the same or one or more may be different from the other(s), and the same is true for the R¹²'s, the R¹³'s, and the R¹⁴'s. Each of R¹¹, R¹², and R¹³ is either H, alkyl, alkenyl, aryl, arylalkyl, alkoxy, alkylthio, alkenoxy, alkenylthio, aryloxy, arylthio, alkyl interrupted by one or more oxa (—O—), alkenyl interrupted by one or more oxa (—O—), alkyl interrupted by one or more thia (—S—), alkenyl interrupted by one or more thia (—S—), aryloxyalkyl, alkoxyaryl, aminoalkyl, aminoalkenyl, aminoaryl, aminoarylalkyl, hydroxyalkyl, hydroxyalkenyl, hydroxyaryl, or hydroxyarylalkyl, provided only that these groups that do not interfere with complexation and that they are not combined in a manner that results in a chemically unstable configuration. Here again, the alkyl, alkenyl and aryl groups, or portions of groups, in the foregoing list can also be substituted with one or more halogen atoms. R¹⁴ in Formula II is defined as H, hydroxy, amino, alkyl, alkyl interrupted by oxa (—O—), alkoxy, aryl, aryloxyalkyl, or alkoxyaryl, or any of these groups in which the alkyl and aryl portions are substituted with one or more halogen atoms. Again, the R¹⁴ groups are selected such that they do not interfere with complexation and are not combined in a manner that results in a chemically unstable configuration.

Further in Formula II, all X's on any single molecule may be the same or one or more may be different from the other(s), and each is either alkyl, alkenyl, aryl, arylalkyl, alkoxy, alkylthio, alkenoxy, alkenylthio, aryloxy, arylthio, alkyl interrupted by oxa, alkenyl interrupted by oxa, alkyl interrupted by thia, alkenyl interrupted by thia, aryloxyalkyl, alkoxyaryl, aminoalkyl, aminoalkenyl, aminoaryl, aminoarylalkyl, hydroxyalkyl, hydroxyalkenyl, hydroxyaryl, or hydroxyarylalkyl, or halogen-substituted versions of any of the preceding radicals, or any of the following:

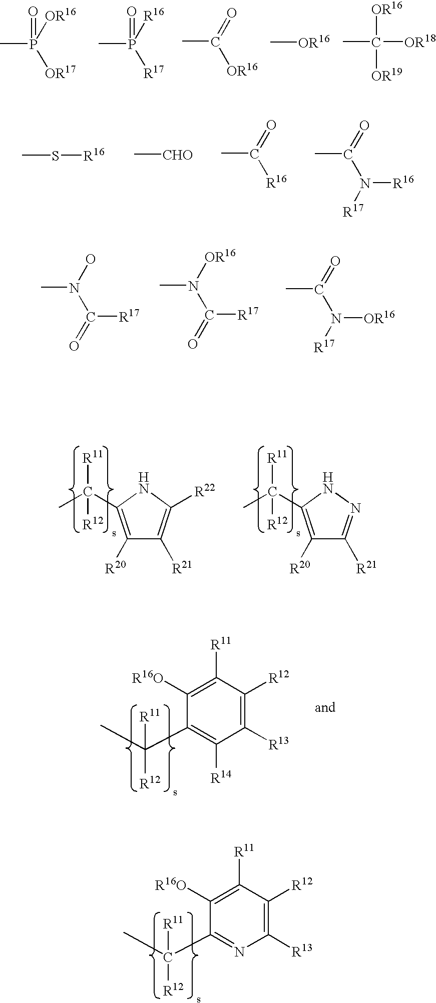

In these formulas, R¹¹, R¹², R¹³ and R¹⁴ may be the same or different on any single radical or on any single molecule, and each has the same definition as that given above for R¹¹, R¹², and R¹³. Also in these formulas, R¹⁶ and R¹⁷ are the same or different on any single radical or molecule, and each is either H, alkyl, or aryl. Alternatively, R¹⁶ and R¹⁷ may be combined to form a ring structure. The groups R¹⁸ and R¹⁹ are likewise the same or different on any single radical or molecule, and are either H, alkyl, aryl, alkoxy, alkyl interrupted by oxa, aryloxyalkyl, or alkoxyaryl, or halogen-substituted versions of these radicals. The groups R²⁰, R²¹ and R²² are likewise the same or different on any single radical or molecule, and are either H, alkyl, alkenyl, aryl, arylalkyl, alkoxy, alkylthio, alkenyloxy, allkenylthio, aryloxy, aminoalkyl, aminoalkenyl, aminoaryl, aminoarylakyl, hydroxyalkyl, hydroxyalkenyl, hydroxyaryl, or hydroxyarylalkyl. The index s is an integer of 1 to 3.

This preferred class further include dimers of the structures of Formula I that are formed by the covalent attachment of two structures of the Formula. Still further members of this class include physiological salts of any of the structures described above.

The terms used in connection with these formulas have the same meaning here as they have in the chemical industry among those skilled in the art. The term “alkyl” thus encompasses both straight-chain and branched-chain groups and includes both linear and cyclic groups. The term “alkenyl” refers to unsaturated groups with one or more double bonds and includes both linear and cyclic groups. The term “aryl” refers to aromatic groups or one or more cycles.

For all groups defined above, those which are useful in the present invention are those that do not impair or interfere with the formation of the chelate complexes. Within this limitation, however, the groups may vary widely in size and configuration. Preferred alkyl groups are those having 1 to 8 carbon atoms, with 1 to 4 carbon atoms more preferred. Prime examples are methyl, ethyl, isopropyl, n-propyl, and tert-butyl. Preferred alkenyl groups are vinyl and allyl, particularly vinyl. Preferred aryl groups are phenyl and naphthyl, particularly phenyl. Preferred arylalkyl groups are phenylethyl and benzyl, and of these, benzyl is the most preferred. Preferred cycloalkyl groups are those with 4 to 7 carbon atoms in the cycle, with cycles of 5 or 6 carbon atoms particularly preferred. Preferred halogen atoms are chlorine and fluorine, with fluorine particularly preferred.

Further preferred embodiments of the present invention are as follows. In Formula I, m, n, and p are preferably each 2, and q is preferably 1. In Formula II, r is preferably zero, and in the radicals shown under the definition of X of Formula II, s is preferably 1 or 2, and most preferably 1. Preferred groups for R¹ are H, alkyl, alkenyl, aryl, aralkyl, and those of Formula II, while preferred groups for R² and R³ are H, alkyl, alkenyl, aryl, and aralkyl. Within Formula II, preferred groups for R¹¹, R¹², and R¹³ are H, alkyl, alkenyl, aryl, and arylalkyl, and preferred groups for R¹⁴ are H, hydroxyl, amino, and alkyl. Preferred groups for X are alkyl, alkenyl, aryl, arylalkyl, and the following radicals:

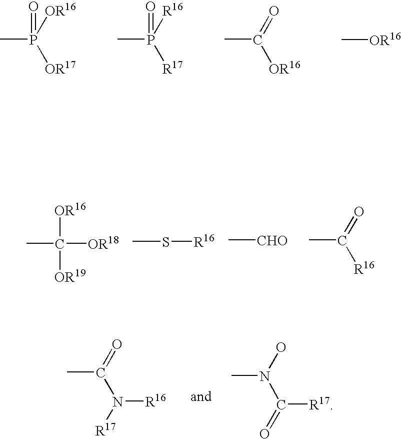

Groups for X that are more preferred are alkyl, alkenyl, aryl, arylalkyl, and the following radicals:

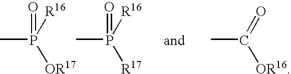

In the radical formulas shown immediately above, preferred groups for R¹⁵, R¹⁶, R¹⁷ and R¹⁸ are H and C₁-C₄ alkyl.

As noted above, this invention resides in the administration of complexes of these chelators with alkaline earth metal cations. In view of their enhanced activity, this invention is of greatest interest in the use of the alkaline earth metal cations magnesium and calcium, with calcium the most preferred.

Preferred chelators for use in this invention are those with molecular weights that do not exceed 20000. More preferred are those whose molecular weight is from about 200 to about 1800, and the most preferred are those whose molecular weight is from about 400 to about 1100.

The first transition series metals that form complexes with the chelators of this invention can assume any of the various oxidation states in which these metals are known to exist in ionic or combined form. Thus, for example, the chelators of this invention can form complexes with ferric ion or ferrous ion, and cupric ion or cuprous ion.

Pharmaceutical compositions containing the ligands described herein are prepared and with according to standard techniques. The pharmaceutical compositions can be administered parenterally, i.e., intraarticularly, intravenously, subcutaneously, or intramuscularly. Suitable formulations for use in the present invention are found in Remington's Pharmaceutical Sciences, Mack Publishing Company, Philadelphia, Pa., 17th ed. (1985).

The pharmaceutical compositions of this invention will generally contain the complexes described above plus a pharmacologically acceptable carrier, preferably an aqueous carrier. A variety of aqueous carriers can be used, such as water, buffered water, 0.9% isotonic saline, and the like. These compositions can be sterilized by conventional, well known sterilization techniques, or may be sterile filtered. The resulting aqueous solutions can be packaged for use as is, or lyophilized, and if lyophilized, the lyophilized preparation will be combined with a sterile aqueous solution prior to administration. The compositions may contain pharmaceutically acceptable auxiliary substances as required to approximate physiological conditions, 0 such as pH adjusting and buffering agents, tonicity adjusting agents, wetting agents and the like, for example, sodium acetate, sodium lactate, sodium chloride, calcium chloride, calcium hydroxide, sorbitan monolaurate, and triethanolamine.

The concentration of the complex in the pharmaceutical composition can vary widely, i.e., from less than about 0.05%, usually at or at least about 2-5% to as much as 10 to 30% by weight, and will be selected primarily by fluid volumes, viscosities, and other such parameters, in accordance with the particular mode of administration selected.

The complexes and pharmaceutical compositions of this invention are useful as means of enhancing the biological activity of cyclic polyaza chelators that have high affinity and specificity for cations of first transition series metals, and therefore for the treatment of any physiological condition that is mitigated by a reduction in the bioavailability of these cations. Included among these conditions are ischemia and ischemia-reperfusion injury, either of which may be the result of any of a variety of procedures or physiological conditions. Included among these procedures and conditions are cardiopulmonary bypass surgery, vascular surgery, and tissue transplants. Also included are ischemic stroke, seizure, trauma, heart attack and arrhythmia. The complexes of this invention are generally useful in providing neuroprotection and cardioprotection in a patient in need of such protection.

The methods of this invention can be performed on a variety of subjects, preferably mammalian subjects such as humans, non-human primates, and dogs, cats, cattle, horses, goats, and sheep, i.e., domestic animals and livestock.

The foregoing description and the following examples are offered primarily for illustration and are not intended to limit scope of the invention. It will be readily apparent to those of ordinary skill in the art that the substances, compositions, methods of formulation, and methods of administration can be further modified or substituted in various ways without departing from the spirit and scope of the invention.

### EXAMPLE 1

This example illustrates the synthesis of chelators (ligands) which are useful in the present invention. Section 1.1 illustrates the synthesis of polyaza bases. Section 1.2 illustrates the synthesis of alkylating groups. Section 1.3 illustrates the preparation of chelating agents from alkylation of polyaza bases.

In all examples reactions were carried out in common solvents, compounds were purified by routine methodology and identity was established by proton NMR. In some cases identity was further verified by elemental analysis, mass spectroscopy, C-13 or P-31 NMR, or by synthesis of the identical compound by an independent alternate synthesis route.

### 1.1 Synthesis of Polyaza Bases

Ethylene diamine (1.1.0), diethylene triamine (1.1.1), triethylenetetramine (1.1.2), 1,4,7-triazacyclononane (1.1.3), 1,4,7,10-tetraazacyclododecane (1.1.4), 1,4,8,11-tetraazacyclotetradecane (1.1.5), and 1,5,9,13-tetraazacyclohexadecane (1.1.6) and the corresponding hydrohalide salts were either obtained from commercial sources or were synthesized employing established methods and were used directly in the syntheses of chelators (ligands) described in section 1.3. Additional polyaza bases were synthesized as described herein.

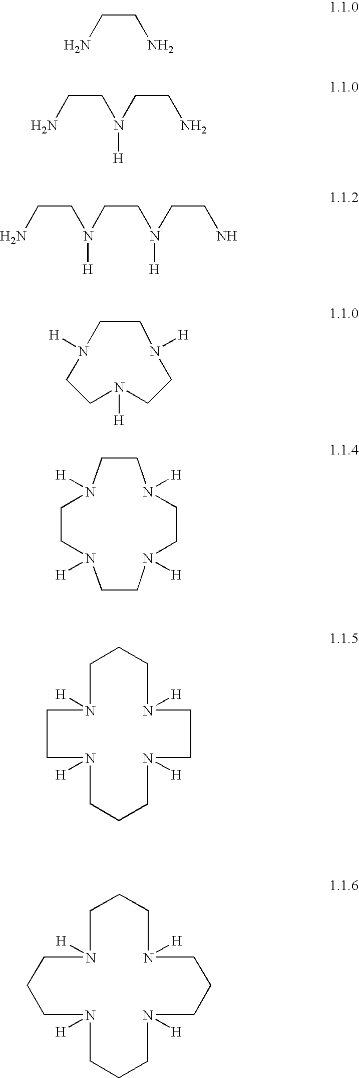

### 1.1.7: 2,6-Diethyl-1,4,7-triazacyclononane Trihydrobromide

2-(p-Toluenesulfonylamino)-1-(p-toluenesulfonyloxy)butane (1.1.8) and ammonium hydroxide were reacted to form 2-(p-toluenesulfonamino)-1-aminobutane (1.1.9). This was reacted with 2-(p-toluenesulfonylamino)-1-(p-toluene-sulfonyloxy)butane (1.1.8) and potassium carbonate. The 3,7 bis(p-toluene-sulfonylamino)-5-azanonane (1.1.10) product was purified by chromatography and reacted with p-toluenesulfonyl chloride to obtain the corresponding tri-p-toluene-sulfonyl compound 3,7 bis(p-toluenesulfonylamino)-5-(p-toluenesulfonyl-5-aza-nonane (1.1.11). This was purified by chromatography and reacted with 2.2 equivalents of sodium amide in DMF and then with 1,2-di(p-toluene-sulfonyloxy)ethane (1.1.12). The 2,6-diethyl-1,4,7-tris(p-toluenesulfonyl) triazacyclononane (1.1.13) that was obtained following purification was heated in a solution of HBr in acetic acid to remove the p-toluenesulfonyl groups and form the titled compound (1.1.7).

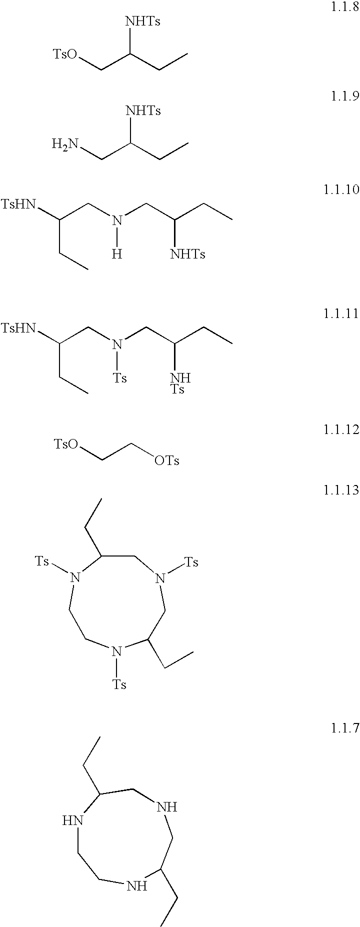

### 1.1.14: 1,4,7-Triazabicyclo[7.4.0]tridecane Trihydrobromide

1,2-trans-bis(p-Toluenesulfonylamino)cyclohexane (1.1.15) was treated with NaH in DMSO. 1-(p-Toluenesulfonylamino)-2-(p-toluenesulfonyl)ethane (1.1.16) was added to obtain 1-(p-toluenesulfonylamino)-2-[N-p-toluenesulfonyl-N-(2-p-toluenesulfonylaminoethyl)]aminocyclohexane (1.1.17). This was separated and reacted with NaH and 1,2-di(p-toluenesulfonyloxy)ethane (1.1.12) was added. The 2,3-butano-N,N,′,N″-tris(p-toluenesulfonyl)-1,4,7-triazacyclononane (1.1.18) obtained was purified by chromatography. The p-toluenesulfonyl groups were removed by reaction in HBr/acetic acid and the 2,3-butano-1,4,7-triazacyclononane trihydrobromide (1.1.14) product precipitated from solution as the hydrobromide salt.

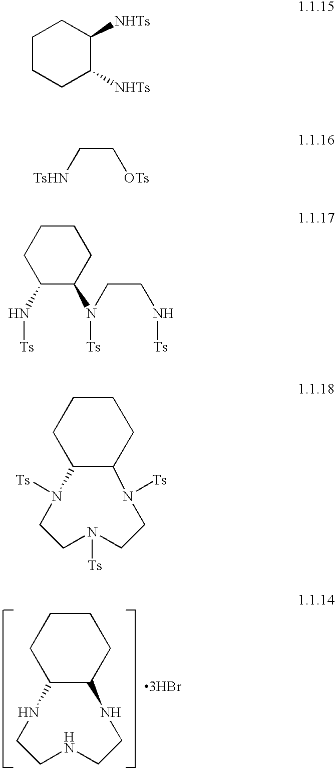

### 1.1.19: 1,3-Bis (1,4,7-triazacyclononane)propane

N,N′-bis(p-Toluenesulfonyl)-1,4,7-triazacyclononane (1.1.20) was prepared by reacting (1.1.3) with two equivalents of p-toluenesulfonyl chloride. Two equivalents of N,N′-bis(p-toluenesulfonyl)-1,4,7-triazacyclononane (1.1.20) hydrobromide were reacted with one equivalent of 1,3-diiodopropane in acetonitrile with excess potassium carbonate. 1,3-bis[N,N′-bis(p-Toluenesulfonyl)-1,4,7-triazacyclononane propane (1.1.21) was isolated and purified by chromatography. The p-toluenesulfonyl groups were removed using sulfuric acid and HBr to yield the title compound (1.1.19).

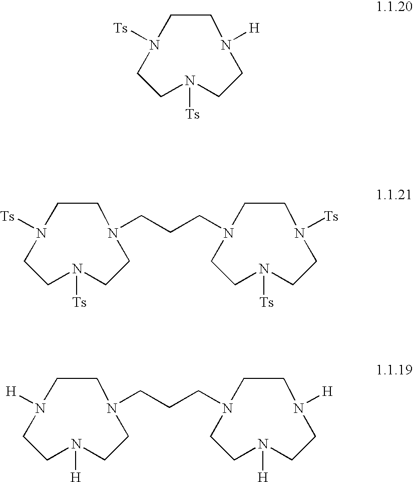

### 1.1.22 1,4,7,10-Tetraazabicyclo[5.5.2]tetradecane

1,4,7,10-Tetraazadodecane (1.1.4)trihydrobromide in acetonitrile with potassium carbonate was reacted with glyoxal to form 1,4,7,10-tetraazatetracyclo-[5,5,2,04,13,010,14]tetradecane (1.1.23). Following separation the pure product was obtained by low pressure distillation. This was dissolved in acetonitrile and benzylbromide was added to form 1,7-dibenzylonium-1,4,7,10-tetraaza-tetracyclo[5,5,2,04,13,010,14]tetradecane (1.1.24). Following recrystallization from ethanol this was reacted with sodium borohydride. HCl was added, followed by water and NaOH, and the product extracted with chloroform. Following evaporation of solvent the solids were dissolved in methanol and HBr was added to obtain 1,7-dibenzyl-1,4,7,10-tetraazabicyclo [5.5.2]tetradecane (1.1.25) as the hydrobromide salt. This was dissolved in water and reduced using H₂ and a Pd-C catalyst to remove the benzyl groups. Purification of the title compound was by crystallization of the hydrobromide salt. The base form was obtained by low pressure distillation following addition of base.

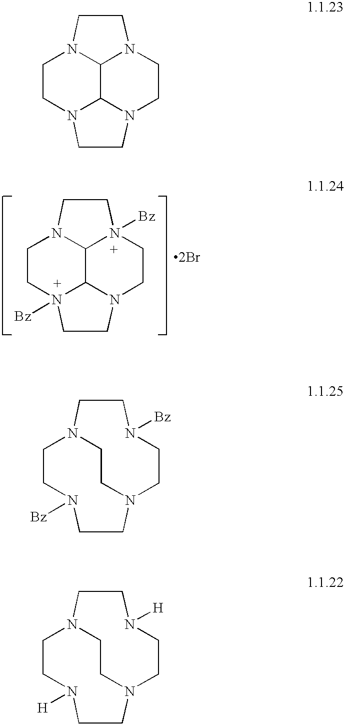

### 1.1.26 1,4,7,10,13-Pentaazabicyclo[8.5.2]heptadecane

To 1,8-bis(p-toluenesulfonyloxy)-3,6-bis(p-toluenesulfonyl)-3,6-diaza-octane (1.1.27) was added 1,4,7-triazacyclononane (1.1.3) in acetonitrile with potassium bicarbonate to obtain 4,7-bis (p-toluenesulfonyl)-1,4,7,10,13-penta-azabicyclo [8.5.2](1.1.28) heptadecane. The title compound was purified and the p-toluenesulfonyl groups were removed by treatment in sulfuric acid. Purification was done by low pressure distillation.

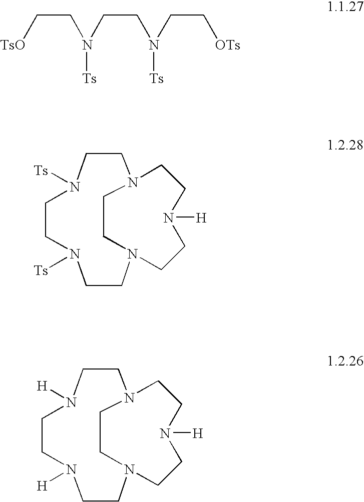

### 1.1.29 1,2-Bis(1,4,7-triazabicyclononane-1-yl)ethane

A mixture of N,N′-bis(p-toluenesulfonyl)-1,4,7-triazabicyclonone hydrobromide (1.1.13.33), ethylene glycol di-p-toluenesulfonyl or dibromoethane and excess of potassium carbonate in acetonitrile was refluxed overnight. The reaction mixture was added to water and extracted with methylene chloride. The tetratosylated product (1.1.30) was purified by chromatography. It was suspended in 70% H₂SO₄ and heated at 150° C. for 15 hours. The reactions cooled to room temperature and then 62% HBr solution was added. The white precipitate was collected and washed with ethanol, then redissolved in water and filtered from tars. The water was made basic and the title compound (1.1.29) was extracted with chloroform.

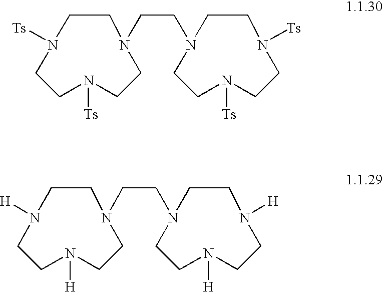

### 1.2 Synthesis of Alkylating Groups for Alkylation of Polyaza Bases to Form Chelators Described in Example 1.3.

### 1.2.1 Preparation of Glycidyl Ethers

Glycidyl tosylate (R, S or d,l) (1.2.1.0) was reacted in the appropriate alcohol solvent employing catalytic amounts of conc. H₂SO₄ or equivalent amounts of tetrafluoroboranetherate. The 1-alkyloxy-2-hydroxy-3-p-toluenesulfonyloxypropane (1.2.1.1) product was reacted in ether with BuLi to yield the title epoxide. The following compounds were prepared in this manner.

### 1.2.1.0 Glycidyl Tosylate (R,S or d,l; Commercially Available)

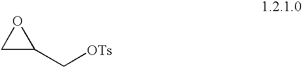

### 1.2.1.1 1-Alkyloxy-2-hydroxy-3-p-toluenesulfonyloxypropane

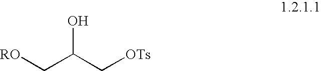

### 1.2.1.2 d,l-Glycidyl-isopropyl Ether (Commercially Available).

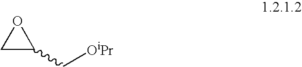

### 1.2.1.3 (2R) Glycidyl-isopropyl Ether

### 1.2.1.4 (2S) Glycidyl-isopropyl Ether

### 1.2.1.5 d,l-Glycidyl-t-butyl Ether

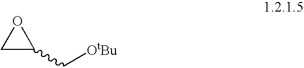

### 1.2.1.6 (2R) Glycidyl-t-butyl Ether

### 1.2.1.7 d,l-Glycidyl Allyl Ether

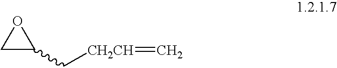

### 1.2.1.8 d,l-Glycidyl Phenyl Ether

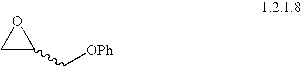

### 1.2.2 Preparation of 2,2-Dialkoxymethylene Oxiranes and Spiro-Oxiranes

3-Chloro-2-chloromethyl-1-propane (1.2.2.0) was reacted with the corresponding sodium alkylate or disodium dialkylate either using the same alcohol or dialcohol as solvent or using an inert solvent. The ether product was purified by distillation or chromatography. Epoxidation was performed using meta-chloroperbenzoic acid in halogenated solvent. The following compounds were prepared in this manner.

### 1.2.2.0 3-Chloro-2-chloromethyl-1-propene (Commercially Available)

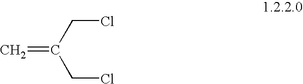

### 1.2.2.1. 2,2-Bis-ethoxymethyl Oxirane

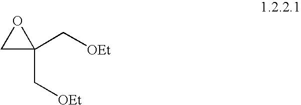

### 1.2.2.2 2,2-Bis-methoxymethyl Oxirane

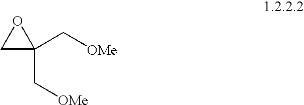

### 1.2.2.3 2,2-Bis-isopropyloxymethyl Oxirane

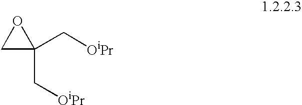

### 1.2.2.4 2,2-Bis-difurfuryloxymethyl Oxirane

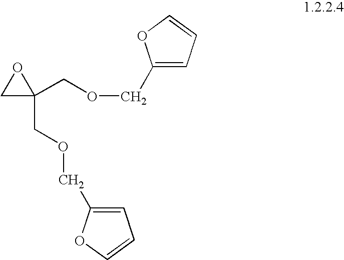

### 1.2.2.5 2, 2-Bis(hydroxymethyl)oxirane

From 2-methylidene-1,3-dihydroxypropenediol (commercially available).

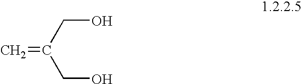

### 1.2.3 Preparation of Oxiranespiro-3-(1,5-Dioxacycloalkanes)

Various dry glycols in DMF were reacted with NaH and 3-chloromethyl-1-propane (1.2.2.0) was added to the resulting reaction mixture. Following completion of the reaction the solvents were removed and the product purified by low pressure distillation. The purified product in dichloroethane was reacted with m-chloroperbenzoic acid to form the corresponding epoxide. Following workup, the epoxide product was purified by distillation. The following compounds were prepared in this manner.

### 1.2.3.1 Oxiranespiro-3-(1,5-dioxacycloheptane)

(From ethylene glycol)

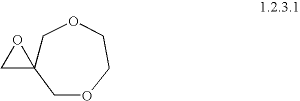

### 1.2.3.2 Oxiranespiro-3-(1,5-dioxa-7,7-dimethylcyclooctane)

(From 2,2-dimethyl propylene glycol)

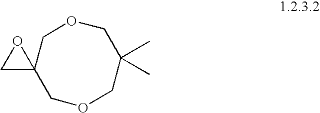

### 1.2.3.3 Oxiranespiro-3-(1,5-dioxa-6-methylcycloheptane)

(From 1,2-dihydroxy propane)

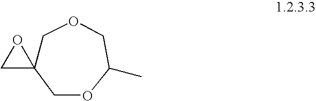

### 1.2.3.4 Oxiranespiro-3-(1,5-dioxa-6,6,7,7-tetramethylcycloheptane)

[From 2,3-dihydroxy-2,3-dimethyl butane (pinacol)].

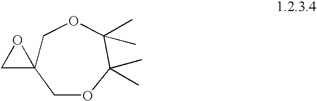

### 1.2.3.5 Oxiranespiro-3-(benzo[b]-1,5-dioxacycloheptane)

(From 1,2-dihydroxybenzene).

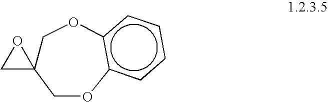

### 1.2.3.6 Oxiranespiro-3-(1,5-dioxacycloctane)

(From 1,3-propanediol)

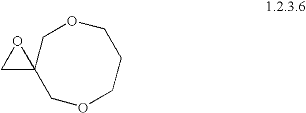

### 1.2.4 Preparation of Miscellaneous Epoxides

### 1.2.4.1 2,2-dimethyl oxirane

(From 2-methyl-1-propene and m-chloroperbenzoic acid

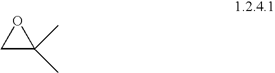

### 1.2.4.2 2-(Isopropyl)-2-[(1-fluoro-1-methyl)ethyl]oxirane

Reaction between 2,4-dimethyl-3-pentanone (1.2.4.3), trimethylsilyl chloride, and base gave 2,4-dimethyl-3-trimethysilyloxy-2-pentene (1.2.4.4) which was reacted with 1-fluoropyridinium triflate (1.2.4.5) to form 2,4-dimethyl-2-fluoro-3-pentanone (1.2.4.6). This product was reacted with (CH₃)₃S(O)^{+I}⁻ to form the title compound (1.2.4.2).

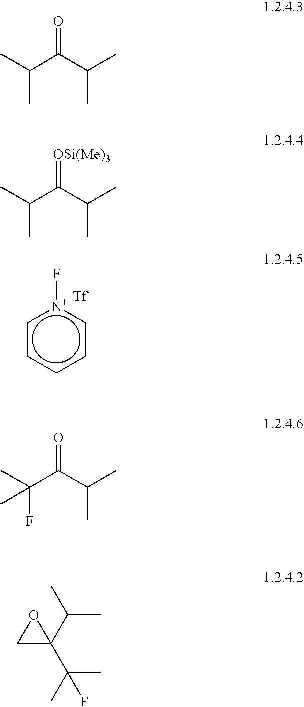

### 1.2.4.7 2,2-Bis-isopropyl Oxirane

(From 2,4-dimethylpentanone using (CH₃)₃S(O)⁺I⁻ as described in 1.2.4.2)

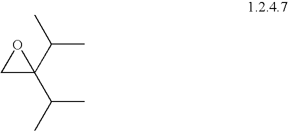

### 1.2.4.8 2-(1-Fluoroethyl)-2-(1-trimethylsilyloxyethyl)oxirane

The title compound was obtained in several steps. DEK was O-silylated using usual procedure. The resulting product was reacted with 1-fluoropyridinium triflate (1.2.4.5) to yield 2-fluoro-3-pentanone (1.2.4.9). After bromination the 2-bromo-4-fuoro-3-pentanone (1.2.4.10) which was obtained was reacted with liquid ammonia to form 2-fluoro-4-hydroxy-3-pentanone (1.2.4.11). The free hydroxyl group was protected with trimethylsilylchloride to form 2-fluoro-4-trimethylsilyloxo-3-pentanone (1.2.4.12). This product was reacted with trimethylsulfoxonium iodide to form the title compound (1.2.4.8).

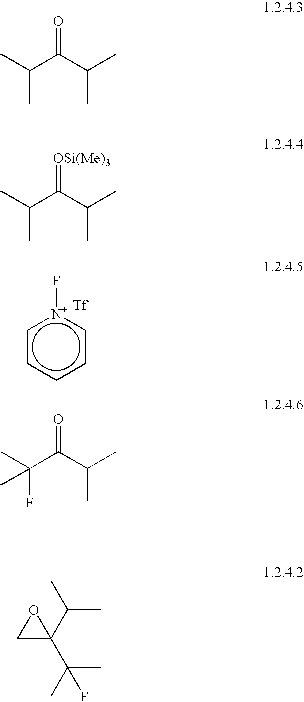

### 1.2.4.13 2-(1-Bromoethyl)-3-methyl oxirane

Bromination of diethyl ketone with bromine gave 2,4-dibromo-3-pentanone (1.2.4.14). This product was reduced with BH₃/THF to form 3-hydroxy-2,4-dibromopentane (1.2.4.15). After treatment with base the title compound (1.2.4.13) was obtained.

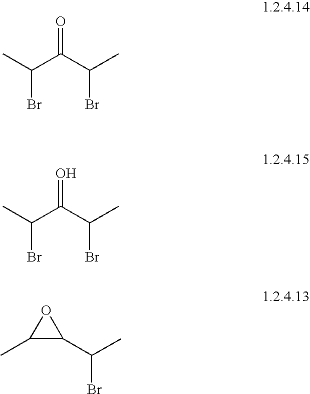

### 1.2.4.16 2-(1-Fluoroethyl)-3-methyl Oxirane

From reaction between diethylketone and trimethylchlorosilane to form 3-trimethylsilyloxy-2-pentene (1.2.4.17). This product was reacted with 1-fluoro-pyridinium triflate (1.2.4.5) to obtain 2-fluoro-3-pentanone (1.2.4.9). After bromination with pyridinium bromide followed by reduction using diborane 2-fluoro-4-bromopentane-3-ol (1.2.4.18) was obtained. Reaction of this product with sodium methylate yielded the title compound (1.2.4.16).

This compound was made also by reacting 2-(1-bromoethyl)-3-methyl oxirane (1.2.4.13) with HF/Py (70%) followed by treatment of the resulting 2-bromo-4-fluoropentan-3-ol (1.2.4.18) with K₂CO₃/MeOH.

### 1.2.4.19 2-(1-Fluoroethyl)-2-(1-methoxyethyl)oxirane

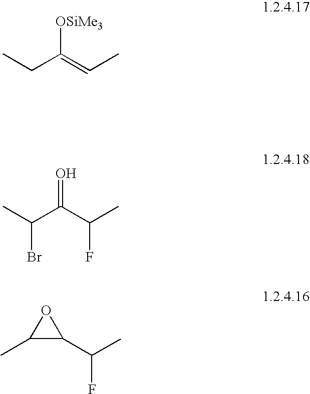

2-(1-Fluoroethyl)-3-methyl oxirane (1.2.4.16) was reacted with methanol/sulfuric acid to obtain 2-fluoro-4-methoxypentane-3-ol (1.2.4.20). This product was reacted with chromic anhydride/pyridine to form 2-fluoro-4-methoxy-pentane-3-one (1.2.4.21) which was then reacted with sodium hydride and trimethylsulfoxonium iodide to obtain the title compound (1.2.4.19).

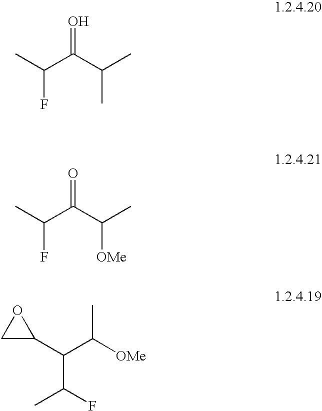

### 1.2.4.22 2-(1-Methoxyethyl)-3-methyl Oxirane

Reaction of 2-(1-Bromoethyl)-3-methyl oxirane (1.2.4.13) with methanol/sulfuric acid formed 2-bromo-3-hydroxy-4-methoxypentane (1.2.4.23). This product was reacted with potassium carbonate in methanol to obtain the title compound (1.2.4.22).

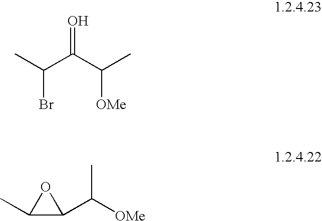

### 1.2.4.24 2-Ethyl-2-(1-methoxyethyl)oxirane

Reaction between diethyl ketone and dimethyl hydrazine gave diethyl ketone-N,N-dimethylhydrazone (1.2.4.25). This product was reacted with dimethyl disulfide/LDA to obtain 2-methylthio-3-pentanone-N,N-dimethyl hydrazone (1.2.4.26). This product was reacted with mercuric chloride followed by cupric chloride to obtain 2-methoxy pentane-3-one (1.2.4.27). Reaction of the latter compound with sodium hydride/DMSO/trimethylsulfonium iodide yielded the title compound (1.2.4.24).

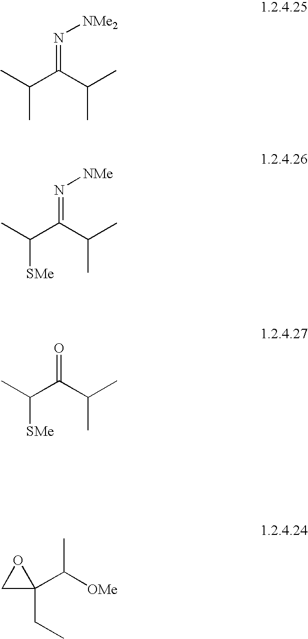

### 1.2.4.28 2-Ethyl-2-(1-trimethylsilyloxyethyl)oxirane

From reaction between 2-bromo-3-pentanone (1.2.4.29) and hydrazine obtained 2-hydroxy-3-pentanone (1.2.4.30). This product was reacted with trimethylchlorosilane/triethylamine to obtain 2-trimethylsilyloxy-3-pentanone (1.2.4.31). This product was reacted with methylenetriphenyl phosphite and butyllithium to obtain 2-ethyl-3-trimethylsilyloxy-1-butene (1.2.4.32). After oxidation with meta-chloroperbenzoic acid in methylene chloride the title compound (1.2.4.28) was obtained.

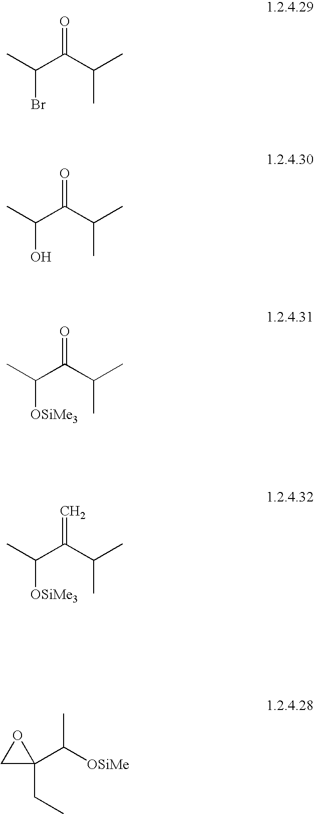

### 1.2.4.33 2,2-Bis(1-fluoroethyl)oxirane

From reaction between 2-(1-Bromoethyl)-3-methyl oxirane (1.2.4.13) and HF/pyridine was obtained 2-bromo-4-fluoro-pentane-3-ol (1.2.4.18). This was reacted with potassium carbonate to obtain 2-(1-fluoroethyl)-3-methyl oxirane (1.2.4.16). This was reacted again with HF/pyridine to obtain 2,4-difluoro-pentane-3-ol (1.2.4.34). After oxidation with chromium trioxide obtained 2,4-difluoro-3-pentanone (1.2.4.35). The epoxide title compound was prepared from the ketone as described for 1.2.4.24.

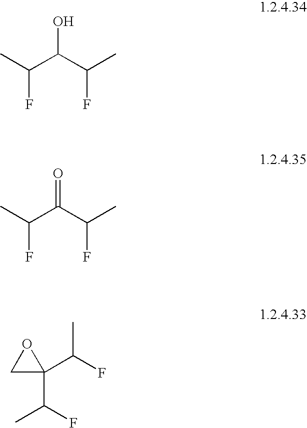

### 1.2.3.36 2,2-Bis-dichloromethyleneoxirane

(From direct epoxidation of 3-chloro-2-chloromethyl-1-propene).

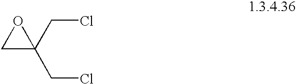

### 1.2.4.37 2,2-Bis(1-methoxyethyl)oxirane

3-Pentanone was brominated to get 2,4-dibromo-3-pentanone (1.2.4.11) using conventional methods. The dibromoketone was reduced with BH₃*THF to the corresponding alcohol (1.2.4.15). This compound was reacted with MeONa in methanol to yield 2-(1-bromoethyl)-3-methyl oxirane (1.2.4.13) which after reaction with MeOH/H₂SO₄ gave 2-bromo-3-hydroxy-4-methoxy pentane (1.2.4.38). This intermediate was reacted again with MeONa in methanol and the resulting 2-(1-methoxyethyl)-3-methyl oxirane (1.2.4.22) was reacted again with MeOH/H₂SO₄ to yield 2,4-dimethoxy-3-hydroxy pentane (1.2.4.39). After oxidation with CrO₃/Py in methylene chloride the resulting ketone was reacted with trimethylsulfoxonium iodide to give the title compound (1.2.4.37).

### 1.2.6.2 1-Bromo-2-t-butyldimethylsilyloxyethane, BrCH₂CH₂Si(^{t}Bu)(CH₃)₂

(From bromoethanol and dimethyl-t-butylsilylchloride)

### 1.2.6.3 5-(p-Toluenesulfonyloxymethylene)-1-benzyloxy-2-pyrrolidone

This compound was prepared in several steps. 4-pentenoic acid (1.2.6.4) was reacted with ethylchloroformate to obtain the active mixed anhydride. To a solution of the mixed anhydride in chloroform was added triethylamine and O-benzylhydroxylamine hydrochloride to obtain O-benzyl-4-pentenohydroxamic acid (1.2.6.5). The double bond was oxidized using osmium tetroxide/N-methyl-morpholine oxide to give the diol (1.2.6.6). The terminal hydroxyl group was then protected with t-butyidimethylsilylchloride in the usual way to yield (1.2.6.7). The secondary hydroxyl group was tosylated using pyridine/p-toluenesulfonyl chloride. Cyclization of (1.2.6.8) to the corresponding pyrrolidone (1.2.6.9) was effected by using sodium carbonate in methanol. The protecting silyl group was removed by treatment with tetraethylammonium fluoride. The title compound (1.2.6.3) was prepared by reacting the latter compound (1.2.6.10) with pyridine/p-toluenesulfonyl chloride.

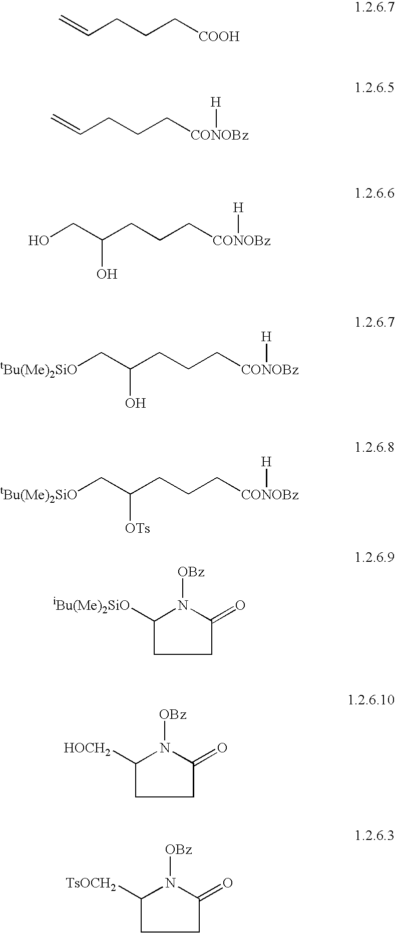

### 1.2.6.11 5-Bromo-1-benzyloxy-2-pyrrolidone

This compound was prepared in several steps. Butyrolactone was reacted with PBr₃/Br₂ to obtain the dibromobutyrylbromide (1.2.6.12). This compound with O-benzylhydroxylamine yielded the protected dibromohydroxamic acid (1.2.6.13). Cyclization was effected by base to give the cyclic protected hydroxamic acid (1.2.6.11).

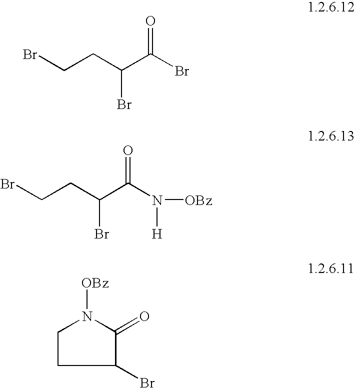

### 1.2.7 Preparation of N-Alkyl-O-benzylchloroacetohydroxamic Acids

This class of compounds was prepared from chloroacetyl chloride and the suitable N-Alkylhydroxylamine followed by O-benzylation with benzyl bromide. In certain instances the O-benzyl alkylhydroxylamine was used as the starting material. O-Methyl chloroacetoxyhydroxamic acid was prepared employing O-methylhydroxylamine as starting material.

### 1.2.7.1 O-Benzyl-N-Methyl chloroacetohydroxamic acid, ClCH₂CON(Me)(OBz)

### 1.2.7.2 O-Benzyl-N-isopropyl-chloroacetohydroxamic Acid, ClCH₂CON(ⁱPr)(OBz)

### 1.2.7.3 O-Benzyl-N-tert-butyl-chloroacetohydroxamic Acid, ClCH₂CON(^{t}Bu)(OBz)

### 1.2.7.4 O-Benzyl chloroacetohydroxamic acid, ClCH₂CONH(OBz)

### 1.2.7.5 0-Methyl chloroacetohydroxamic acid, ClCH₂CONH(OMe)

### 1.3 Synthesis of Chelators (Ligands)

### 1.3.1 Synthesis of Polyaza Ligands with Pendant Arms Containing β-Hydroxy Groups and Their Derivatives

This family of compounds was prepared by reacting polyaza free bases with epoxides or halohydrines in water or alcohol solvents.

### 1.3.1.1 N,N′,N″-Tris(2-hydroxy-3-isopropoxypropyl)-1,4,7-Triazacyclononane

From 1,4,7-triazacyclononane (1.1.3) and d,l-glycidyl isopropyl ether (1.2.1.2).

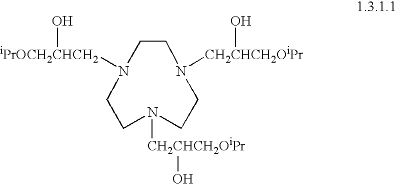

### 1.3.1.2 (R,R,R)N,N′,N″-Tris(2-hydroxy-3-isopropoxypropyl)-1,4,7-triazacyclononane

From 1,4,7-Triazacyclononane (1.1.3) and (2R)glycidyl isopropyl ether (1.2.1.3).

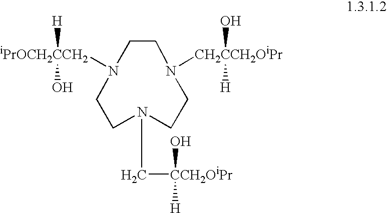

### 1.3.1.3 (S,S,S)N,N′,N″-Tris(2-hydroxy-3-isopropoxypropyl)-1,4,7-triazacyclononane

From 1,4,7-Triazacyclononane and (2S)glycidyl isopropyl ether (1.2.1.4).

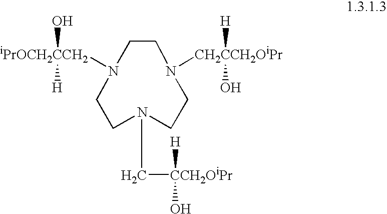

### 1.3.1.4 N,N′,N″-Tris(2-hydroxy-3-t-butoxypropyl)-1,4,7-triazacyclononane

From 1,4,7-Triazacyclononane (1.13) and (d,l)glycidyl-t-Butyl ether (1.2.1.5).

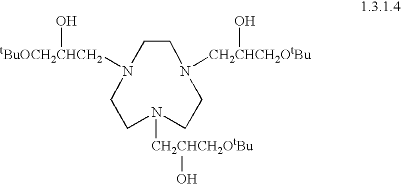

### 1.3.1.5 (R,R,R)N,N′,N″-Tris(2-hydroxy-3-t-butoxypropyl)-1,4,7-triazacyclononane

From 1,4,7-triazacyclononane (1.1.3) and (R)glycidyl t-butyl ether (1.2.1.6).

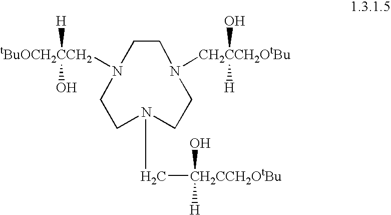

### 1.3.1.6 N,N′,N″-Tris(2-hydroxy-3 methoxypropyl)-1,4,7-triazacyclononane

From 1,4,7-triazacyclononane (1.1.3) and (d,l)glycidyl methyl ether (commercially available).

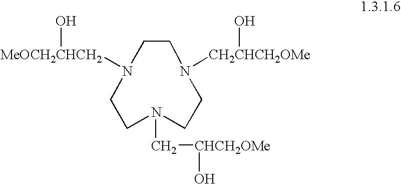

### 1.3.1.7 N,N′,N″-Tris(2,3-dihydroxypropyl)-1,4,7-triazacyclononane

From 1,4,7-triazacyclononane (1.1.3) and 1-bromo-2,3-dihydroxypropane (commercially available) and excess of potassium carbonate or 1-chloro-2,3-dihydroxypropane (commercially available) and base.

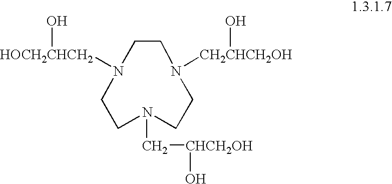

### 1.3.1.8 N,N′,N″-Tris(1-methoxy-2-hydroxy-2-methylpropyl)-1,4,7-triazacyclononane

From 1,4,7-triazacyclononane (1.1.3) and (d, I) 3,3-dimethyl-2-methoxy oxirane (1-methoxy-2-methylpropylene, commercially available).

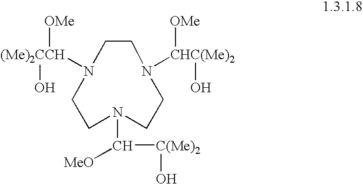

### 1.3.1.9 N,N′,N″-Tris(2-hydroxy-3-allyloxypropyl)-1,4,7-triazacyclononane

From 1,4,7-triazacyclononane (1.1.3) and (d,l)glycidyl allyl ether (1.2.1.7).

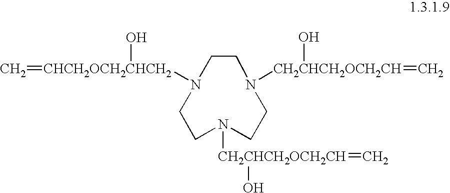

### 1.3.1.10 N,N′,N″-Tris(2-hydroxy-3-phenoxypropyl)-1,4,7-triazacyclononane

From 1,4,7-triazacyclononane (1.1.3) and (d,l)glycidyl phenyl ether (1.2.1.8).

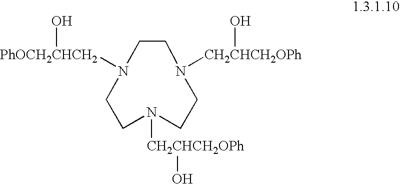

### 1.3.1.11 N,N′,N″-Tris(2-hydroxy-2,2-diethoxymethylene)ethyl-1,4,7-triazacyclononane

From 1,4,7-triazacyclononane (1.1.3) and 2,2-bis-ethoxymethyl oxirane (1.2.2.1).

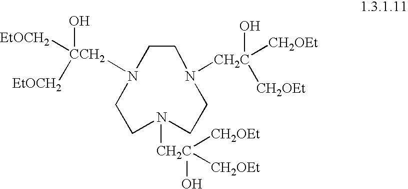

### 1.3.1.12 N,N′,N″-Tris(2-hydroxy-2,2-dimethoxymethyl)ethyl-1,4,7-triazacyclononane

From 1,4,7-triazacyclononane (1.1.3) and 2,2-bis-methoxyoxymethyl oxirane (1.2.2.2).

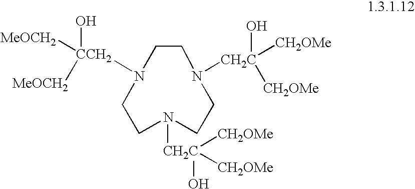

### 1.3.1.13 N,N′,N″-Tri(2-hydroxy-(2,2-diisopropyloxymethyl)ethyl-1,4,7-triazacyclononane

From 1,4,7-triazacyclononane (1.1.3) and 2,2-Bis-Isopropoxymethyl oxirane (1.2.2.3).

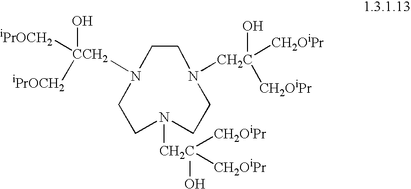

### 1.3.1.14 N,N′,N″-Tris[2-hydroxy-bis(2-furfuryloxymethyl)ethyl]-1,4,7-triazacyclononane

From 1,4,7-triazacyclononane (1.1.3) and 2,2-bis(furfuryloxymethyl)oxirane (1.2.2.4).

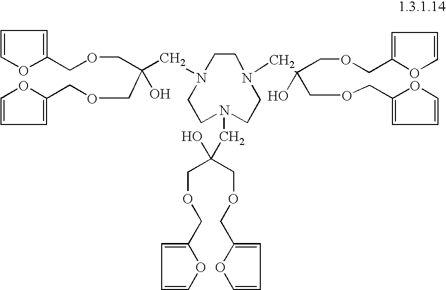

### 1.3.1.15 N, N′,N″-Tris(3-hydroxy-1,5-dioxacycloheptyl-3-methyl)-1,4,7-triazacyclononane

From 1,4,7-triazacyclononane (1.1.3) and oxiranespiro-3-(1,5-dioxacycloheptane) (1.2.3.1).

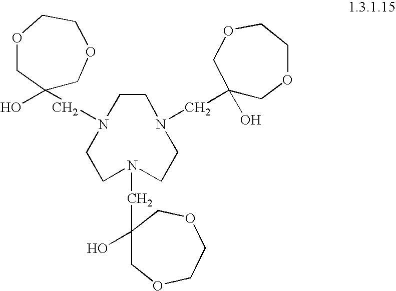

### 1.3.1.16 N, N′,N″-Tris[(3-Hyd roxy-7,7-dimethyl-1,5-dioxacyclooct-3-yl)-methyl]-1,4,7-triazacyclononane

From 1,4,7-triazacyclononane (1.1.3) and oxiranespiro-3-(1,5-dioxa-7,7-dimethylcyclooctane) (1.2.3.2).

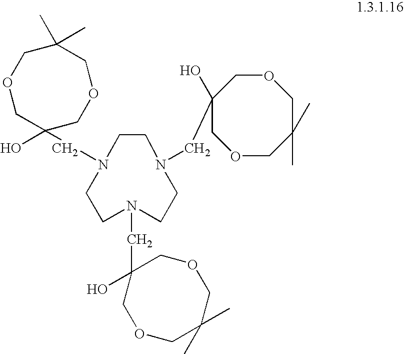

### 1.3.1.17 N,N′,N″-Tris[(3-hydroxy-7-methyl-1,5-dioxacyclohept-3-yl)methyl]-1,4,7-triazacyclononane

From 1,4,7-triazacyclononane (1.1.3) and oxiranespiro-3-(1,5-dioxa-6-methylcycloheptane (1.2.3.3).

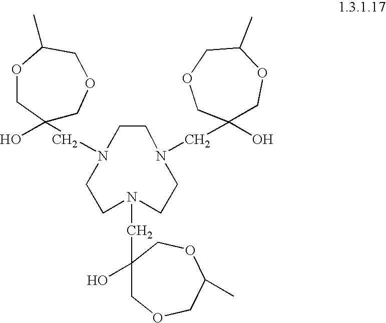

### 1.3.1.18 N,N′,N″-Tris[(3-hydroxy-6,6,7,7-tetramethyl-1,5-dioxacyclohept-3-yl)methyl]-1,4,7-triazacyclononane

From 1,4,7-triazacyclononane (1.1.3) and oxiranespiro-3-(1,5-dioxa-6,6,7,7-tetramethylcycloheptane) (1.2.3.4).

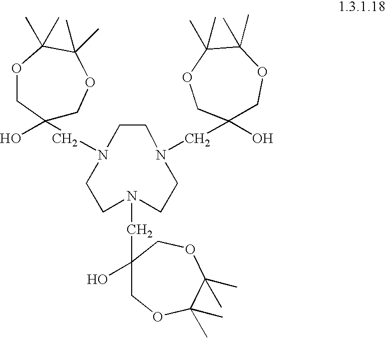

### 1.3.1.19 N, N′,N″-Tris[(3-hydroxy-benzo[b]-1,5-dioxacycloheptyl)methyl]1,4,7-triazacyclononane

From 1,4,7-triazacyclononane (1.1.3) and oxiranespiro-3-(benzo[b]-1,5-dioxacycloheptane) (1.2.3.5).

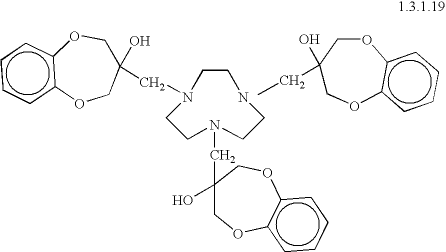

### 1.3.1.20 N,N′,N″-Tris[(3-hydroxy-1,5-dioxacyclooctane-3-yl)methyl]-1,4,7-triazacyclononane

From 1,4,7-triazacyclononane (1.1.3) and oxiranespiro-3-(1,5-dioxacyclooctane) (1.2.3.6).

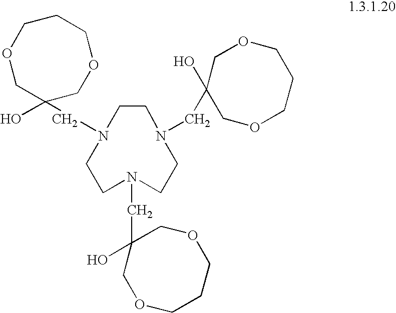

### 1.3.1.21 N,N′,N″-Tris(2-hydroxy-2-methyl propyl)-1,4,7-triazacyclononane

From 1,4,7-triazacyclononane (1.1.3) and 2,2-dimethyl oxirane (1.2.4.1)

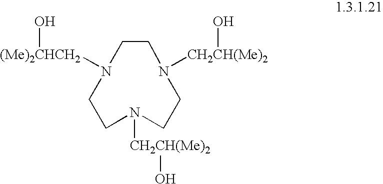

### 1.3.1.22 N,N′,N″-Tris[(4-fluoro-2-hydroxy-3-1-propyl-4-methyl) pentyl]-1,4,7-triazacyclononane

From 1,4,7-triazacyclononane (1.1.3) and 2-isopropyl-2-(1-fluoro-1-methylethyl)oxirane (1.2.4.2).

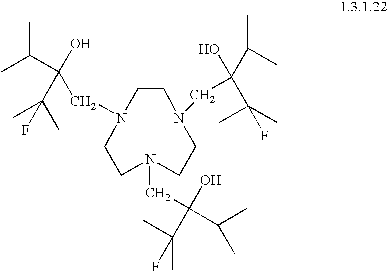

### 1.3.1.23 N, N′,N″-Tris-[2-hydroxy-3-(1-fluoroethyl)-4-hydroxypentyl]-1,4,7-triazacyclononane

From 1,4,7-triazacyclononane (1.1.3) and 2-(1-trimethylsilyloxyethyl)-2-(1-fluoroethyl)oxirane (1.2.4.8).

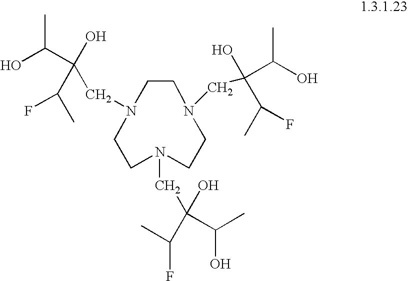

### 1.3.1.24 N,N′,N″-Tris[2-hydroxy-2-(1-fluoroethyl)-2-(1-methoxyethyl)ethyl]-1,4,7-triazacyclononane

From 1,4,7-triazacyclononane (1.1.3) and 2-(1-Fluoroethyl)-2-(1-methoxyethyl)oxirane (1.2.4.19).

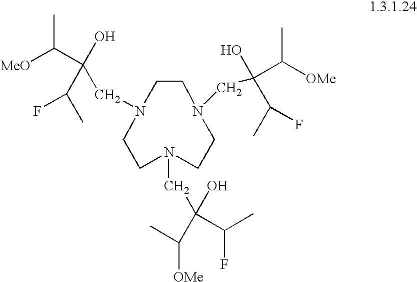

### 1.3.1.25 N,N′,N″-Tris(2-hydroxy-2-ethyl-3-methoxy butyl)-1,4,7-triazacyclononane

From 1,4,7-triazacyclononane (1.1.3) and 2-ethyl-2-(1-methoxyethyl)oxirane (1.2.4.24).

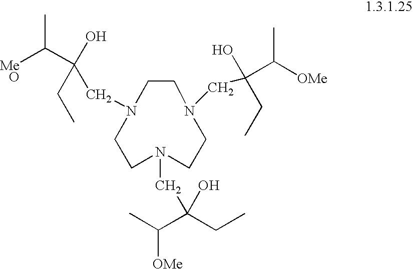

### 1.3.1.26 N, N′,N″-Tris(2,3-dihydroxy-2-ethyl)butyl]-1,4,7-Triazacyclononane

From 1,4,7-triazacyclononane (1.1.3) and 2-ethyl-2-(1-trimethylsilyloxyethyl) oxirane (1.2.4.28).

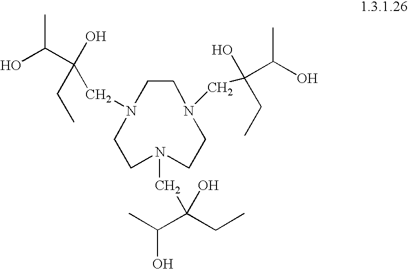

### 1.3.1.27 N,N′,N″-Tris[2-hydroxy-2,2-bis(1-fluoro ethyl)ethyl]-1,4,7-triazacyclononane

From 1,4,7-triazacylononane (1.1.3) and 2,2-bis(1-fluoroethyl)oxirane (1.2.4.33).

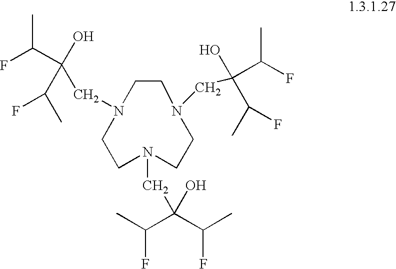

### 1.3.1.28 N,N′,N″-Tris[2-hydroxy-2,2-bis(1-methoxyethyl)ethyl]-1,4,7-triazacyclononane

From 1,4,7-triazacyclononane (1.1.3) and 2,2-(1-methoxyethyl)oxirane (1.2.4.37).

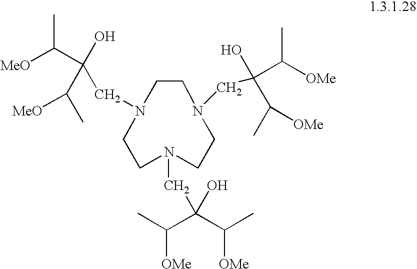

### 1.3.1.29 N,N′,N″-Tris[(3,3-dimethyl-2-hydroxy)butyl]-1,4,7-triazacyclononane

From 1,4,7-Triazacyclononane (1.1.3), 1-Bromo-2-hydroxy-3,3-dimethylbutane (1.2.6.1) and sodium carbonate.

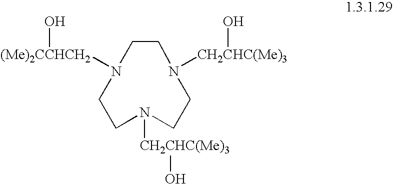

### 1.3.1.30 N,N′,N″-Tris(2-hydroxypropyl)-1,4,7-triazacyclononane

From 1,4,7-triazacyclononane (1.1.3) and propylene oxide.

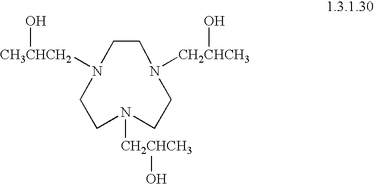

### 1.3.1.31 N,N′,N″-Tris(2,2-dimethoxyethanyl)-1,4,7-triazacyclononane

From 1,4,7-triazacyclononane (1.1.3), 1-chloro-2,2-dimethoxyethane (commercially available) and sodium carbonate.

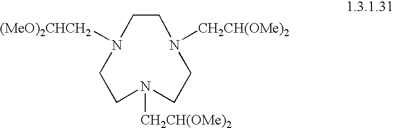

### 1.3.1.32 N,N′,N″-Tris(2-hydroxycyclopentan-1-yl)-1,4,7-triazacyclononane

From 1,4,7-triazacyclononane (1.1.3), 1,2-epoxycyclopentane (commercially available) and sodium carbonate.

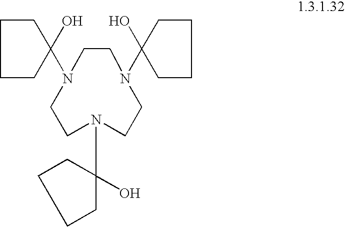

### 1.3.1.33 N,N′,N″-Tris(2-hydroxycyclohexane-1-yl)-1,4,7-triazacyclononane

From 1,4,7-triazacyclononane (1.1.3), 1,2-epoxycyclohexane (commercially available) and sodium carbonate.

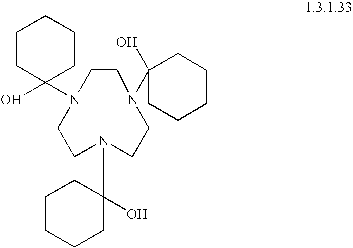

### 1.3.1.34 N,N′,N″-Triallyl-1,4,7-Triazacyclononane

From 1,4,7-triazacyclononane (1.1.3), sodium hydride and allyl bromide.

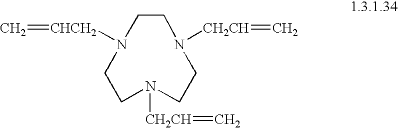

### 1.3.1.35 N,N′,N″-Tris[(3-chloro-2-hydroxy)propyl)]-1,4,7-Triazacyclononane

From N,N′,N″-triallyl-1,4,7-triazacyclononane (1.3.1.34) and aqueous chlorine.

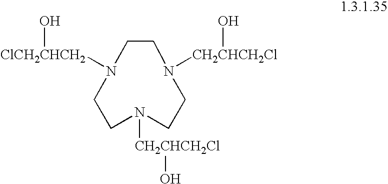

### 1.3.1.36 1,2-Bis-(N,N′-di-2-hydroxyethyl-1,4,7-triazacyclononane-1-yl)ethane

From 1,2-bis-(1,4,7-triazacyclononane-1-yl)ethane polyhydrobromide and ethylene oxide.

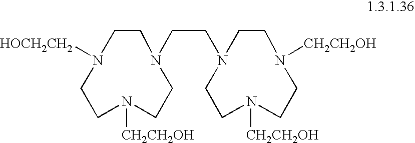

### 1.3.1.37 N,N′,N″,N′″-Tetrakis-(2-hydroxyethyl)-1,4,7,10-tetraazacyclododecane

From 1,4,7,10-Tetraazacyclododecane (1.1.4) and bromoethanol.

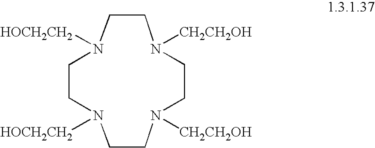

### 1.3.1.38 N,N′,N″,N′″-Tetrakis(2,3-dihydroxypropyl)-11,4,7,10-tetraazacyclotetradecane

From 1,4,7,10-tetraazacyclotetradecane (1.1.4), 1-chloro-2,3-propanediol (commercially available) and base.

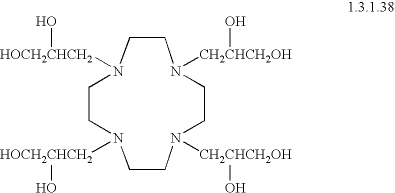

### 1.3.1.39 4,10-Bis(2-Hydroxypropyl)-1,4,7,10-tetraazabicyclo[5.5.2]tetradecane

From 1,4,7,10-tetraazabicyclo[5.5.2]tetradecane (1.1.4) and propylene oxide.

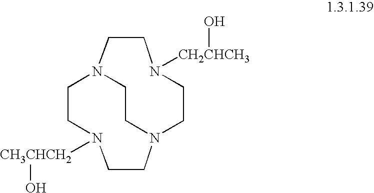

### 1.3.1.40 4,10-Bis-(2-hydroxyethyl)-1,4,7,10-tetraazabicyclo[5.5.2]tetradecane

From 4,10-Bis(dimethoxycarbonylmethyl)-1,4,7,10-tetraazabicyclo[5.5.2]tetradecane (1.3.6.8) and lithium aluminum hydride.

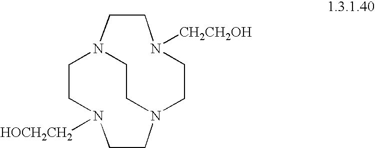

### 1.3.1.41 4,10-Bis[(2-Hydroxy-2-phenyl)ethyl]-1,4,7,10-tetraazabicyclo[5.5.2]tetradecane

From 1,4,7,10-Tetraazabicyclo[5.5.2]tetradecane (1.1.4) and styrene oxide.

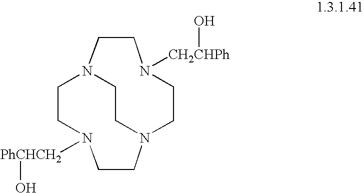

### 1.3.1.42 4,10-Bis-(2,3-dihydroxypropyl)-1,4,7,10-tetraazabicyclo [5.5.2]tetradecane

From 1,4,7,10-tetraazabicyclo[5.5.2]tetradecane (1.1.4) and glycidol.

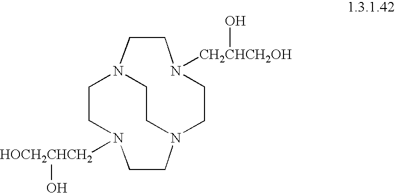

### 1.3.1.43 N,N′,N″,N′″-Tetrakis-(2,3-dihydroxypropyl)-1,4,8,11-tetraazacyclohexadecane

From cyclam (1.1.5) and glycidol.

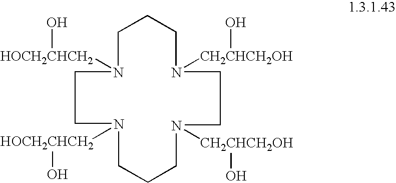

### 1.3.1.44 cis, trans N,N,N′,N′-Tetrakis(2,3-dihydroxypropyl)-1,2-diamino-cyclohexane

From cis,trans 1,2-diaminocyclohexane (commercially available) and glycidol.

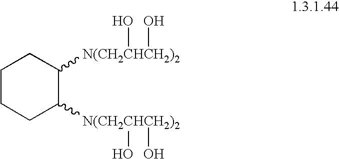

### 1.3.1.45 trans N,N,N′,N′-Tetrakis(2,3-dihydroxypropyl)-1,2-diamino-cyclohexane

From trans-1,2-diaminocyclohexane (commercially available) and glycidol.

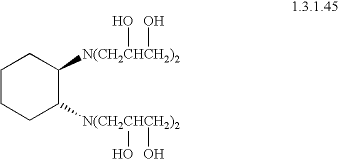

### 1.3.1.51 N,N′,N″-Tris(2,3-diacetoxypropyl)-1,4,7-triazacyclononane

From 3.1.7 and Py/Ac₂O.

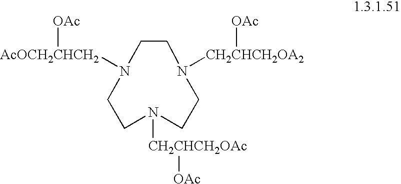

### 1.3.1.52 N,N′,N″-tris(Dimethyl-2,3-isopropylidene propyl)-1,4,7-triazacyclononane

From 1.3.1.7 and 2,2-dimethoxypropane/p-toluenesulfonic acid.

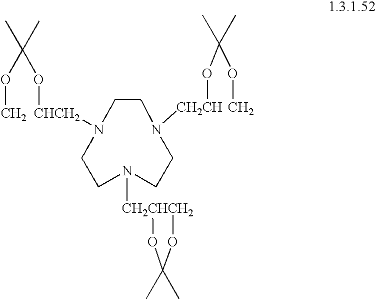

### 1.3.1.53 4,10-(2-Diacetoxyoxypropyl)-1,4,7,10-tetraazabicyclo [5.5.2]tetradecane

From 1.3.1.39 and Py/Ac₂O.

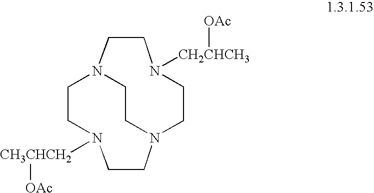

### 1.3.1.54 N,N′,N″-Tris[(2,4-dihydroxy-3-isopropyl-4-methyl)pentyl]-1,4,7-triazacyclononane

From 1,4,7-triazacyclononane (1.1.3) and 2,2-bis(hydroxymethyl)oxirane.

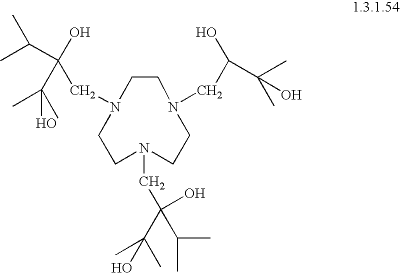

### 1.3.1.55 N, N′,N″-Tris-[2-hydroxy-(2,2-dihydroxymethyl)ethyl]-1,4,7-triazacyclononane

From 1,4,7-triazacyclononane (1.1.3) and 2,2-bis(hydroxymethyl)oxirane (1.2.2.5).

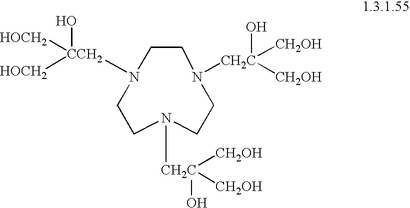

### 1.3.2 Synthesis of Polyaza Ligands With Alkylphosphonate Mono- and Di-Esters Pendant Arms

### 1.3.2.1 Preparation

Chelators having three identical methylene phosphonate diester arms were prepared by reacting the trihydrobromide polyaza bases with formaldehyde and dialkylphosphite. The hexa-ester was hydrolized to the tri-ester by heating with NaOH dissolved in the appropriate alcohol (the same R group as in the dialkylphosphite). In some cases products were obtained by reacting the amine base with haloalkylphosphonates or epoxyphosphonates.

### 1.3.2.1 N,N′,N″-Tris(dibutylphosphorylmethyl)-1,4,7-triazacyclononane

From 1,4,7-triazacyclononane (1.1.3)trihydrobromide, formaldehyde solution and di-n-butyl phosphite (1.2.5.1).

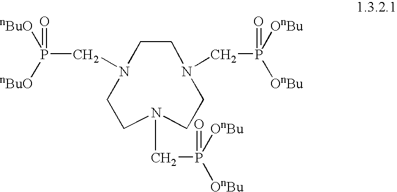

### 1.3.2.2 N,N′,N″-Tris(dihydroxyphosphorylmethyl mono butyl ester)-1,4,7-triazacyclononane

From N,N′,N″-tris(dibutylphosphorylmethyl)-1,4,7-triazacyclononane (1.3.2.1) and KOH/butanol.

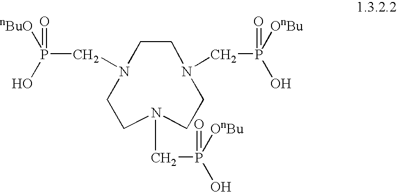

### 1.3.2.3 N,N′,N″-Tris(diethylphosphorylmethyl)-1,4,7-triazacyclononane

From 1,4,7-triazacyclononane (1.1.3)trihydrobromide, formaldehyde solution and diethyl phosphite (commercially available).

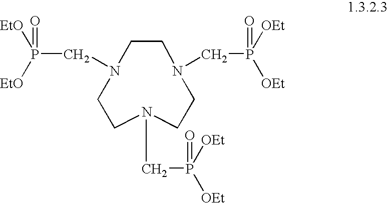

### 1.3.2.4 N,N′,N″-Tris(dihydroxyphosphorylmethyl monoethyl ester)-1,4,7-triazacyclononane

From N,N′,N″-tris(diethylphosphorylmethyl)-1,4,7-triazacyclononane (1.3.2.3) and NaOH/EtOH.

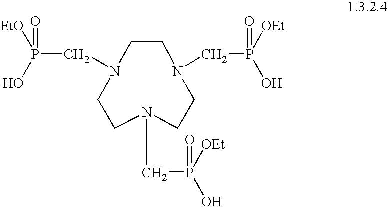

### 1.3.2.5 N,N′,N″-Tris(dioctylphosphophosphorylmethyl)-1,4,7-triazacyclononane

From 1,4,7-triazacyclonane (1.1.3), formaldehyde and dioctylphosphite (1.2.5.2).

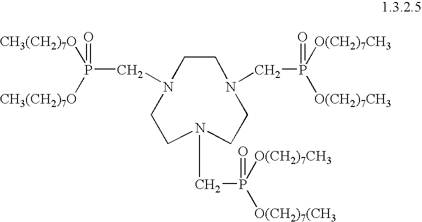

### 1.3.2.6 N,N′,N″-Tris(dihydroxyphosphorylmethyl monooctyl ester)-1,4,7 triazacyclononane

From 1.3.2.5 and NaOH in octyl alcohol.

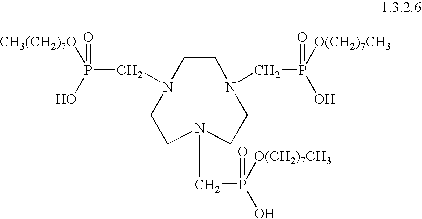

### 1.3.2.7 N, N′,N″-Tris(diisobutylphosphorylmethyl)-1,4,7-triazacyclononane

From 1,4,7-triazacyclononane (1.1.3), formaldehyde and diisobutylphosphite (1.2.5.3).

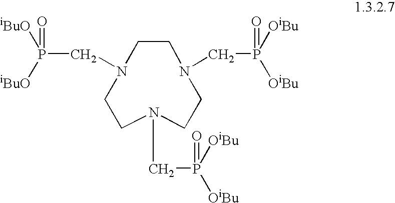

### 1.3.2.8 N,N′,N″-Tris(dihydroxyphosphorylmethyl monoisobutyl ester)-1,4,7-triazacyclononane

From 1.3.2.7 and NaOH in isobutyl alcohol.

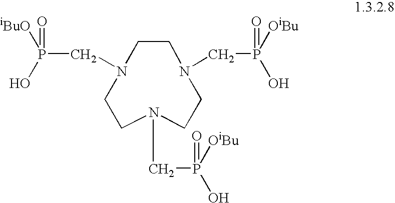

### 1.3.2.9 N,N′,N″-Tris(dibenzylphosphorylmethyl)-1,4,7-triazacyclononane

From 1,4,7-triazacyclononane (1.1.3), formaldehyde and dibenzylphosphite (1.2.5.4).

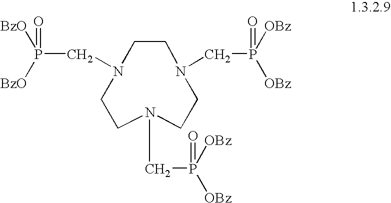

### 1.3.2.10 N,N′,N″-Tris(diethylphosphorylethyl)-1,4,7-triazacyclononane

From 1,4,7-triazacyclononane (1.1.3)trihydrobromide, potassium carbonate and diethyl(2-bromoethyl)phosphonate (1.2.5.5).

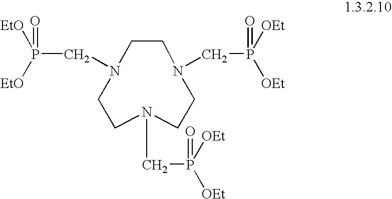

### 1.3.2.11 N,N′,N″,N′″-Tetrakis(diethylphosphorylmethyl)-1,4,7,10-tetraaza-cyclodecane

From 1,4,7,10-tetraazacyclodecane (1.1.4)trihydrobromide, formaldehyde and diethylphosphite (commercially available).

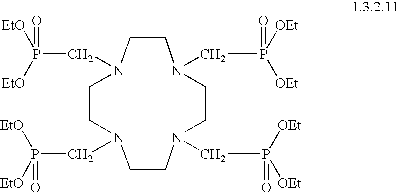

### 1.3.2.12 N,N′,N″,N′″-Tetrakis(diethylphosphorylethyl)-1,4,7,10-tetraaza-cyclododecane

From 1,4,7,10-tetraazacyclododecane (1.1.4)trihydrobromide, potassium carbonate and diethyl(2-bromoethyl)phosphonate (1.2.5.5).

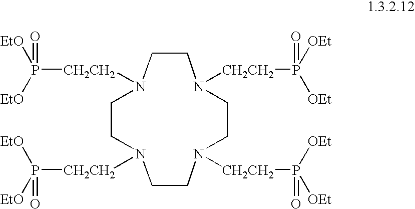

### 1.3.2.13 4,10-Bis(diethylyphosphorylethyl)-1,4,7,10-tetraazabicyclo [5.5.2]tetradecane

From 1,4,7,10-tetraazabicyclo[5.5.2]tetradecane (1.1.20)dihydrobromide, potassium carbonate and diethyl(2-bromoethyl)phosphonate (1.2.5.5).

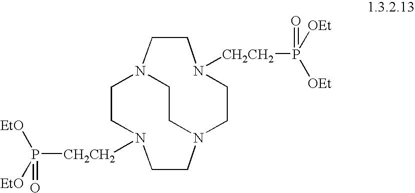

### 1.3.2.14 4,10-Bis(diethylphosphoryl methyl)-1,4,7,10-tetraazabicyclo [5.5.2]tetradecane

From 1,4,7,10-tetraazabicyclo[5.5.2]tetradecane (1.1.20)trihydrobromide, formaldehyde and diethylphosphite (commercially available).

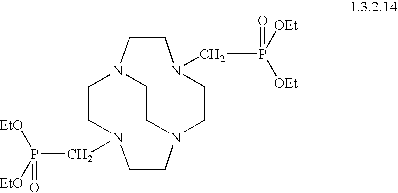

### 1.3.2.15 N,N′,N″-Tris(diethylphosphorylmethyl)-1,4,7,10,13-pentaazabicyclo [8.5.2]heptadecane

From 1,4,7,10,13-pentaazabicyclo[8.5.2]heptadecane (1.1.25), formaldehyde and diethylphosphite (commercially available).

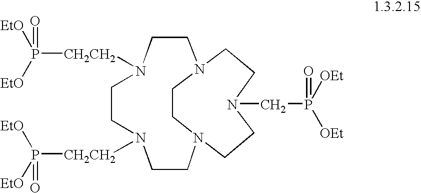

### 1.3.3 Synthesis of Polyaza Ligands with Identical Alkylphosphonic Acid Pendant Arms

These compounds were prepared by either hydrolizing the ester groups of the compounds described under 1.3.2, or from the polyaza base, formaldehyde and phosphorous acid.

### 1.3.3.1 1,2-Bis(N,N′-bis(dihydroxyphosphrylmethyl)-1,4,7-triazacyclononan-1-yl)ethane

From 1,2-bis-(1,4,7-triazacyclononan-1-yl)ethane (1.1.28), formaldehyde and phosphorous acid.

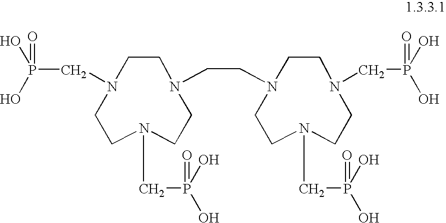

### 1.3.3.2 1,2-Bis(N,N′-bis(dihydroxyphosphorylmethyl)-1,4,7-triazacyclononan-1-yl)propane

From 1,2-bis-(1,4,7-triazacyclononan-1-yl)propane (1.1.19), formaldehyde and phosphorous acid.

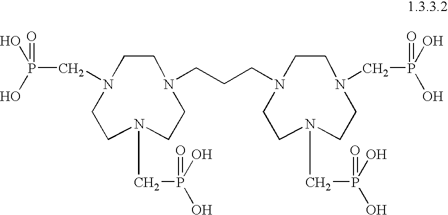

### 1.3.3.3 4,10-Bis(dihydroxyphosphorylmethyl)-1,4,7,10-tetraazabicyclo[5.5.2]tetradecane

From 1,4,7,10-tetraazabicyclo[5.5.2]tetradecane (1.1.20)trihydrobromide, formaldehyde and phosphorous acid.

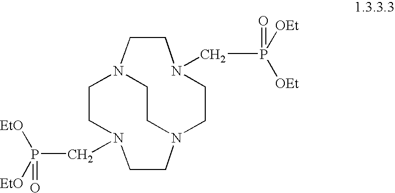

### 1.3.3.4 4,7,13-Tris(dihydroxyphosphorylmethyl)-1,4,7,10,13-pentaazabicyclo[8.5.2]heptadecane

From hydrolysis of 1,4,7,13-tris(diethylphosphorylmethyl)-1,4,7,10,13-pentaazabicyclo[8.5.2]heptadecane (1.3.2.15) by HCl.

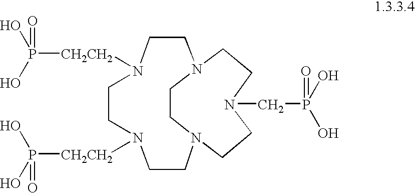

The following compounds were prepared from the corresponding diesters by hydrolysis with HCl:

### 1.3.3.5 N,N′,N″-Tris(dihydroxyphosphorylethyl)-1,4,7-triazacyclononane

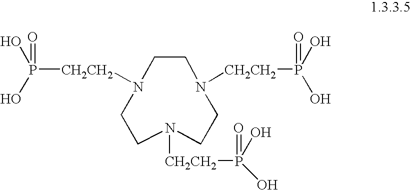

### 1.3.3.6 N,N′,N″,N′″-Tetrakis(dihydroxyphosphorylethyl)-1,4,7,10-tetraazacyclododecane

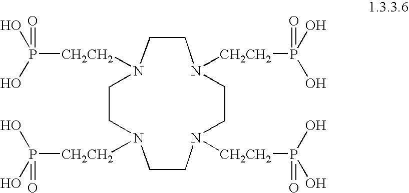

### 1.3.3.7 4,10-Bis(dihydroxyphosphorylethyl)-1,4,7,10-tetraazabicyclo[5.5.2]tetradecane

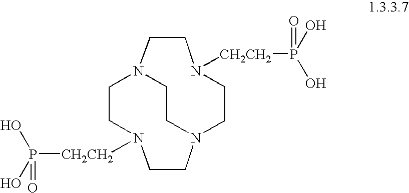

### 1.3.4 Synthesis of Polyaza Ligands with Pendant Arms Containing Phosphonate Esters and Acids with Alpha Substituent Groups

Alkyl or aryl groups α to the phosphonate moiety were prepared by alkylation of the corresponding ligand in the form of its dialkylphosphonate.

### 1.3.4.1 N,N′,N″-Tris[α-dihydroxyphosporyl-α-benzyl)methyl]-1,4,7-triazacyclononane

From N,N′,N″-Tris[(α-diethylphosporyl-α-benzyl)methyl]-1,4,7-triazacyclononane (U.S. Pat. No. 5,380,515) and trimethylsilyl iodide.

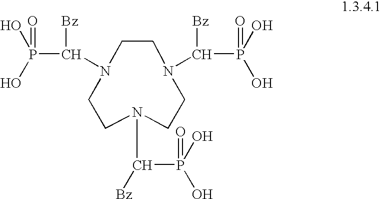

### 1.3.4.2 N,N′,N″-Tris{[(diethylphosphoryl)-α-hydroxylethyl}-1,4,7-triazacyclononane

From 1,4,7-triazacyclononane (1.1.3) and 2-diethylphosphoryl oxirane (1.2.5.7).

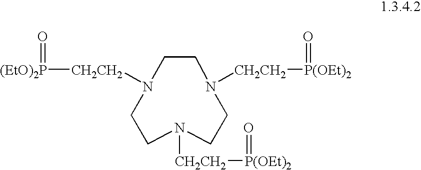

### 1.3.4.3 N,N′,N″-Tris[dihydroxyphosphoryl-α-hydroxy)ethyl]-1,4,7-triazacyclononane

From 1.3.4.2 and HCl.

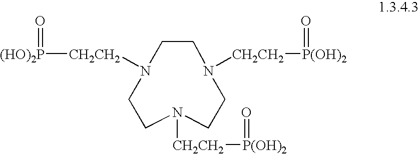

### 1.3.5 Synthesis of Polyaza Ligands with Pendant Arms Containing Hydroxamate Groups

These compounds were prepared by reacting 1,4,7-tetraazacyclononane (1.1.3)trihydrobromide with a N-alkyl-O-benzyl chloroacetohydroxamic acid in the presence of a base. The free hydroxamic acid was obtained by removing the benzyl protecting group by hydrogenolysis.

### 1.3.5.1 N,N′,N″-Tris[(N-methyl-N-benzyloxycarbamoyl)methyl]1,4,7-triazacyclononane

From 1,4,7-triazacyclononane, sodium carbonate and O-benzyl-N— methyl chloroacetohydroxamate (1.2.7.1).

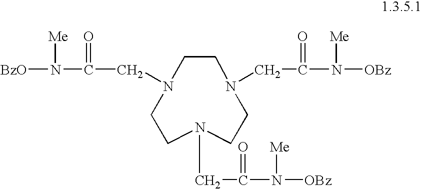

### 1.3.5.2 N,N′,N″-Tris[(N-methyl-N-hydroxycarbamoyl)methyl]-1,4,7-triazacyclononane

From N, N′,N″-tris[(N-methyl-N-benzyloxycarbamoyl)methyl]-1,4,7-triazacyclononane (1.3.5.1) and H₂ and Pd/C.

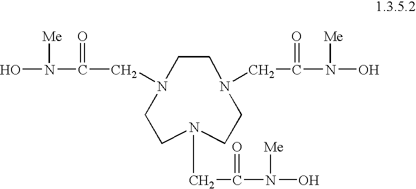

### 1.3.5.3 N,N′,N″-Tris[(N-isopropyl-N-benzyloxycarbamoyl)methyl]-1,4,7-triazacyclononane

From 1,4,7-triazacyclononane trihydrobromide and chloroaceto-N-isopropyl-O-benzyl hydroxamate (1.2.7.2).

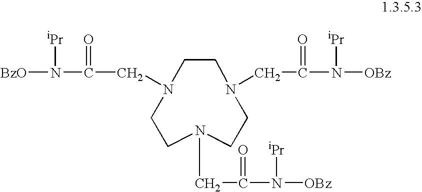

### 1.3.5.4 N,N′,N″-Tris[(N-isopropyl-N-hydroxycarbamoyl)methyl]-1,4,7-triazacyclononane

From 1.3.5.3 and H₂ and Pd/C.

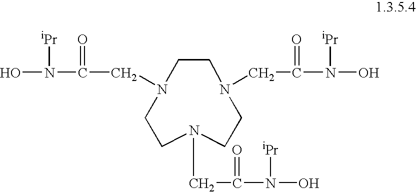

### 1.3.5.5 N,N′,N″-Tris[(N-t-butyl-N-benzyloxycarbamoyl)methyl]-1,4,7-triazacyclononane

From 1,4,7-triazacyclononane trihydrobromide and chloroaceto-N-t-butyl-O-benzyl hydroxamate (1.2.7.3).

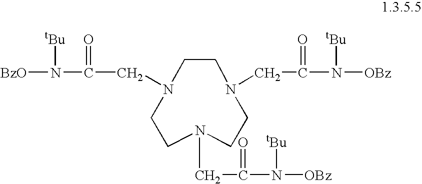

### 1.3.5.6 N,N′,N″-Tris[(N-t-butyl-N-hydroxycarbamoyl)methyl]-1,4,7-triazacyclononane

From 1.3.5.5, H₂ and Pd/C.

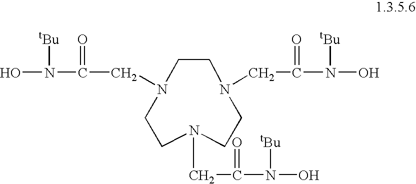

### 1.3.5.7 N,N′,N″-Tris[(N-benzyloxycarbamoyl)methyl]-1,4,7-triazacyclononane

From 1,4,7-triazacyclononane (1.1.3)trihydrobromide and chloroaceto-O-benzyl hydroxamate (1.2.7.4).

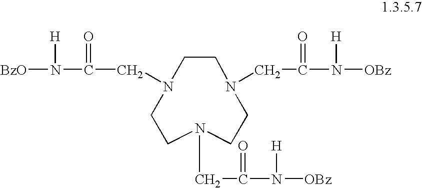

### 1.3.5.8 N, N′,N″-Tris[(N-hydroxycarbamoyl)methyl]-1,4,7-triazacyclononane

From 1.3.5.7 and H₂ and Pd/C.

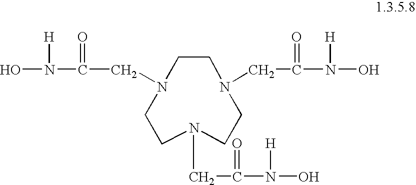

### 1.3.5.9 N,N′,N″-Tris[(N-methoxycarbamoyl)methyl]-1,4,7-triazacyclononane

From 1,4,7-triazacyclononane (1.1.3)trihydrobromide and chloroaceto-O-methyl hydroxamate (1.2.7.5).

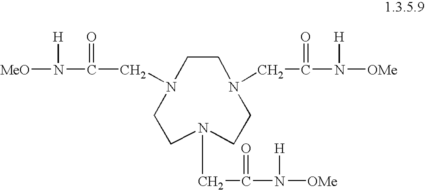

### 1.3.5.10 4,10-Bis[(N-benzyloxycarbamoyl-N-methyl)methyl]-1,4,7,10-tetraazabicyclo[5.5.2]tetradecane

From 1,4,7,10-tetraazabicyclo[5.5.2]tetradecane (1.1.20)dihydrobromic acid, sodium carbonate and chloroaceto-O-benzyl hydroxamate (1.2.7.4).

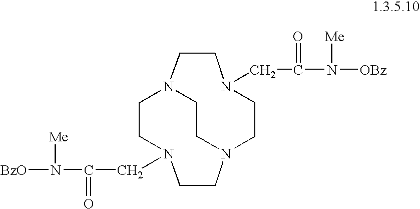

### 1.3.5.11 4,10-Bis[(N-hydroxycarbamoyl-N-methyl)methyl]-1,4,7,10-Tetraaza-bicyclo[5.5.2]tetradecane

From 1.3.5.10 and H₂ and Pd/C.

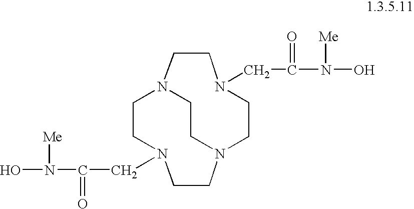

### 1.3.5.12 N,N′,N″-Tris[(1-benzyloxy-2-pyrrolidone-5-yl)methyl]-1,4,7-triazacyclononane

From 1,4,7-triazacyclononane (1.1.3), 5-(p-toluenesulfonyloxymethyl)-1-benzyloxy-2-pyrrolidone (1.2.6.3) and base.

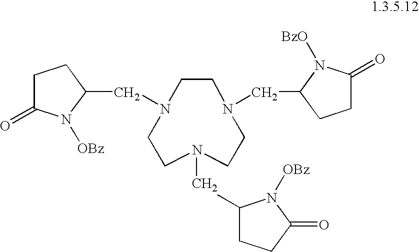

### 1.3.5.13 N,N′,N″-Tris[(1-oxy-2-pyrrolidone-5-yl)methyl]-1,4,7-triazacyclononane

From 1.3.5.12 and Pd/C (5%) and H₂.

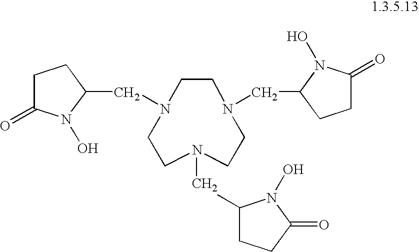

### 1.3.5.14 N,N′,N″-Tris(1-benzyloxy-2-pyrrolidone-5-yl)-1,4,7-triazacyclononane

From 1,4,7-triazacyclononane (1.1.3), 5-bromo-1-benzyloxy-2-pyrrolidone (1.2.6.1 1) and base.

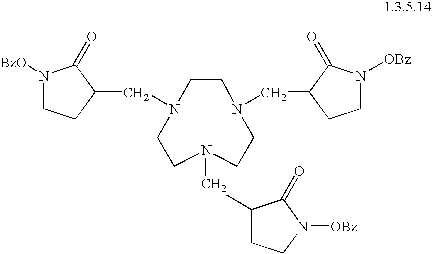

### 1.3.5.15 N,N′,N″-Tris(1-oxy-2-pyrrolidone-5-yl)-1,4,7-triazacyclononane

From 1.3.5.14 and Pd/C (5%) and H₂.

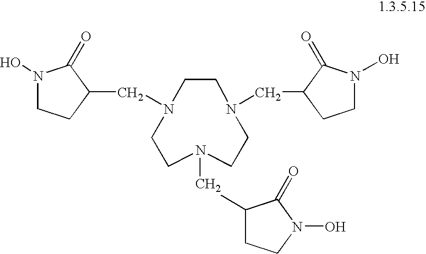

### 1.3.6 Synthesis of Polyaza Ligands with Pendant Arms Containing Carboxyl Groups And The Corresponding Esters

Compounds were prepared by reacting polyaza bases with either halo carboxylic acids or by reductive alkylation with aldo or keto acids. The esters were prepared either by reacting directly with halo carboxylic acid esters or by reaction of the free acid with SOCl₂/alcohol.

### 1.3.6.1 N,N′,N″-Tris(carboxymethyl)-1,4,7-triazacyclononane

From 1,4,7-triazacyclononane (1.1.3), glyoxylic acid and H₂/Pt.

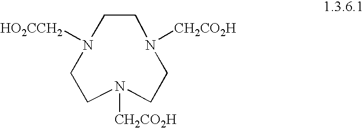

### 1.3.6.2 N,N′,N″-Tris(methoxycarbonylmethyl-1,4,7-triazacyclononane

From N,N′,N″-tris(carboxymethyl)-1,4,7-triazacyclononane in methanol and SOCl₂.

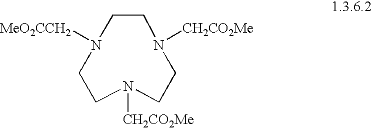

### 1.3.6.3 N,N′,N″-Tris(α-methylcarboxymethyl)-1,4,7-Triazacyclononane

From 1,4,7-triazacyclononane (1.1.3), pyruvic acid and H₂/Pt.

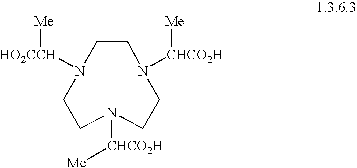

### 1.3.6.4 N,N′,N″-Tris(methoxycarbonylmethyl-1,4,7-triazabicyclo-[7.4.0]tridecane

From 1,4,7-Triazabicyclo[7.4.0]tridecane hydrobromide (1.1.14), glyoxylic acid and H₂/PtO₂ in methanol.

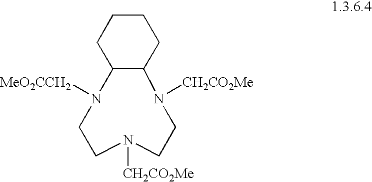

### 1.3.6.5 N-(α-methylcarboxymethyl)-1,4,7-triazabicyclo[7.4.0]tridecane

From 1,4,7-triazabicyclo[7.4.0]tridecane (1.1.14), pyruvic acid and H₂/PtO₂.

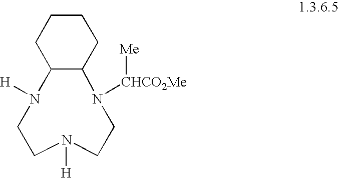

### 1.3.6.6 N,N′,N″-Tris(ethoxycarbonylmethyl)-1,4,7-triazacyclo[7.4.0]tridecane

From 1,4,7-triazabicyclo[7.4.0]tridecane (1.1.14), sodium methoxide and ethyl bromoacetate.

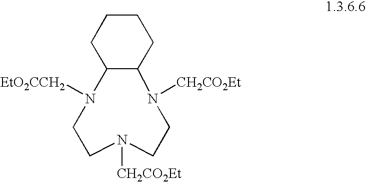

### 1.3.6.7 1,2-Bis-(4,7-carboxymethyl-1,4,7-triazacyclononan-1-yl)ethane

From 1,2-Bis(1,4,7-triazacyclononan-1-yl)ethane (1.1.28), chloroacetic acid and NaOH.

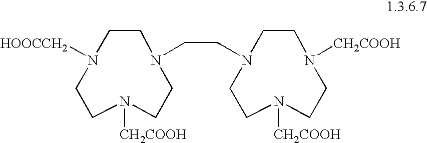

### 1.3.6.8 4,7-Bis(carboxymethyl)-1,4,7,10-tetraazabicyclo[5.5.2]tetradecane

From 1,4,7,10-tetraazabicyclo[5.5.2]tetradecane (1.1.22), chloroacetic acid and NaOH.

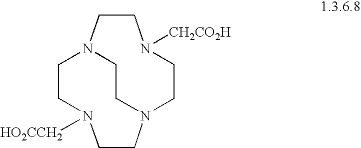

### 1.3.6.9 4,7-Bis(methoxycarboxymethyl)-1,4,7,10-tetraazabicyclo[5.5.2]tetradecane

From 4,7-bis(carboxymethyl)-1,4,7,10-tetraazabicyclo[5.5.2]tetradecane (1.1.20) in MeOH/H₂SO₄.

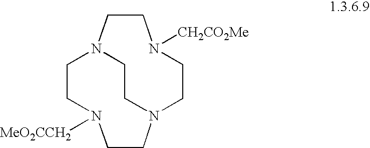

### 1.3.6.10 N,N′,N″-Tris(carboxyethyl)-1,4,7-triazacyclononane

From 1,4,7-triazacyclononane (1.1.3), 3-chloropropionic acid and base.

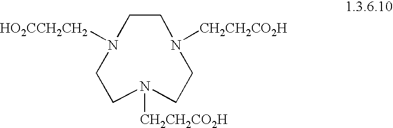

### 1.3.6.11 4,10-Bis(ethoxycarboxymethyl)-1,4,7,10-tetraazabicyclo[5.5.2]tetradecane

From 1,4,7,10-tetraazabicyclo[5.5.2]tetradecane (1.1.20) and ethyl acrylate.

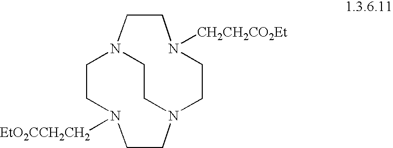

### 1.3.6.12 4,10-Bis(carboxymethyl)-1,4,7,10-tetraazabicyclo[5.5.2]tetradecane

From 1.3.6.11 by acid hydrolysis.

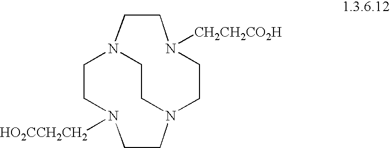

### 1.3.6.13 N,N′,N″-Tris(ethoxycarbonylmethyl)-1,4,7-triazacyclononane

From 1,4,7-triazacyclononane (1.1.3), ethyl bromoacetate and base.

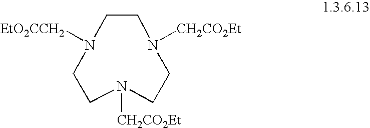

### 1.3.6.14 1,2-Bis-(4,7-methoxycarbonylmethyl-1,4,7-triazacyclononan-1-yl)-ethane

From 1,2-bis-(4,7-carboxymethyl-1,4,7-Triazacyclononan-1-yl)ethane (1.3.6.7), MeOH/SOCl₂.

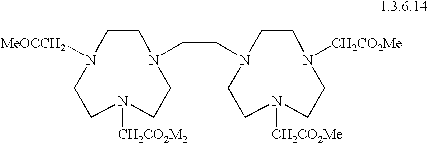

### 1.3.7 Synthesis of Polyaza Ligands with Pendant Arms Containing Aldehyde or Ketone Groups

### 1.3.7.1 N,N′,N″-Tris(2,2-dimethoxyethyl)-1,4,7-triazacyclononane

From 1,4,7-triazacyclononane (1.1.3), 1-chloro-2,2-dimethoxyethane (commercially available) and sodium carbonate.

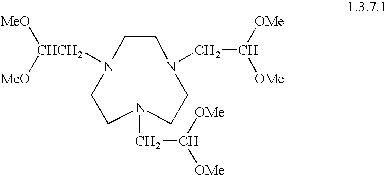

### 1.3.7.2 N,N′,N″-Tris-(3,3-dimethyl-2-oxo-butyl)-1,4,7-triazacyclononane

From 1,4,7-triazacyclononane (1.1.3), bromomethyl t-butyl ketone (commercially available) and sodium carbonate.

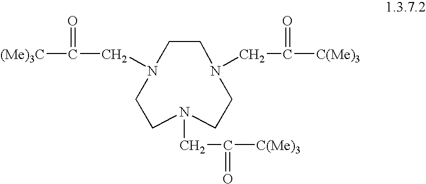

### 1.3.8 Synthesis of Polyaza Ligands with Pendant Arms Containing Pyrrole Groups

### 1.3.8.1 N,N′,N″-Tris(-pyrrol-2-yl-methyl)-1,4,7-triazacyclononane

From 1,4,7-triazacyclononane (1.1.3), pyrrole-2-carboxaldehyde (commercially available) and H₂/PtO₂.

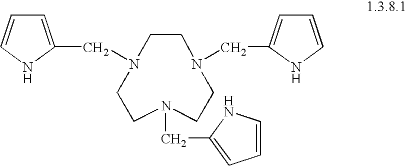

### 1.3.9 Synthesis of Polyaza Ligands with Pendant Arms Containing Amine Groups

### 1.3.9.1 N,N′,N″-Tris(2-p-toluenesulfonyloxyethyl)-1,4,7-triazacyclononane

From 1,4,7-triazacyclononane (1.1.3), 2-(p-toluenesulfonylamino)-1-(p-toluenesulfonyloxy)ethane (1.1.16) and base.

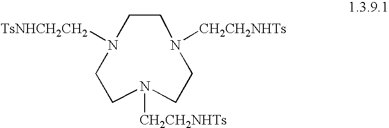

### 1.3.9.2 N,N′,N″-Tris(2-aminoethyl)-1,4,7-triazacyclononane

From 1.3.9.1 and HBr/acetic acid.

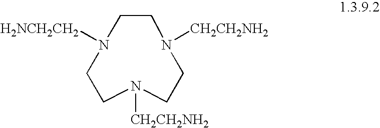

### 1.3.10 Synthesis of Polyaza Ligands with Pendant Arms Containing Amide Groups

### 1.3.10.1 N,N′,N″-Tris(methylcarboxamide)-1,4,7-triazacyclononane

From N, N′,N″-Tris-(methoxycarboxymethyl)-1,4,7-triazacyclononane (1.3.6.2) and ammonia.

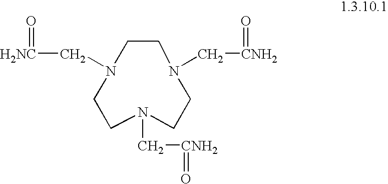

### 1.3.10.2 N,N′,N″-Tris[-N-n-butyl(methylcarboxamide)]-1,4,7-triazacyclononane

From N,N′,N″-Tris-(methoxycarboxymethyl)-1,4,7-triazacyclononane (1.3.6.2) and butylamine.

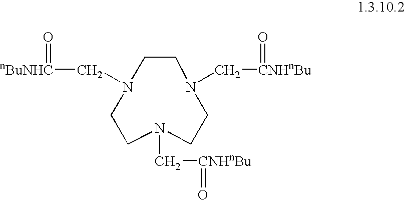

### 1.3.10.3 N,N′,N″-Tris[-N-n-phenyl(methylcarboxamide)]-1,4,7-triazacyclononane

From 1,4,7-triazacyclononane (1.1.3), N-phenylchloroacetamide (prepared from aniline and chloroacetyl chloride) and excess sodium carbonate.

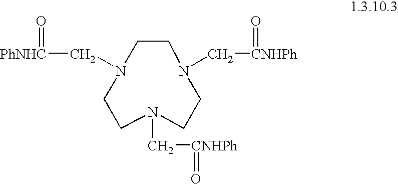

### 1.3.11 Synthesis of Polyaza Ligands with Pendant Arms Containing Phenolic Groups

### 1.3.11.1 4,7-Di-(2-hydroxy-benzyl)-1,4,7,10-tetraazabicyclo[5.5.2]tetradecane

From 1,4,7,10-tetraazabicyclo[5.5.2]tetradecane (1.1.20), salicylaldehyde (excess) and H₂/PtO₂.

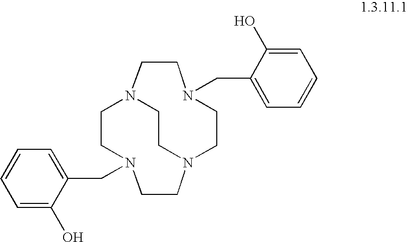

### 1.3.1 1.2 4-(2-hydroxy-benzyl)-1,4,7,10-tetraazabicyclo[5.5.2]tetradecane

From 1,4,7,10-tetrazabicyclo[5.5.2]tetradecane (1.1.20), salicylaldehyde (1.5 equivalents) and H₂/PtO₂.

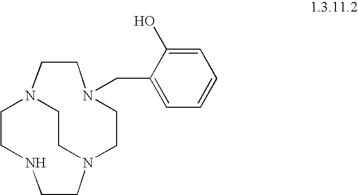

### 1.3.11.11 N,N′,N″-Tris(2-hydroxybenzyl)-1,4,7-triazacyclononane

From 1,4,7-triazacyclononane, salicylaldehyde and H₂/PtO₂.

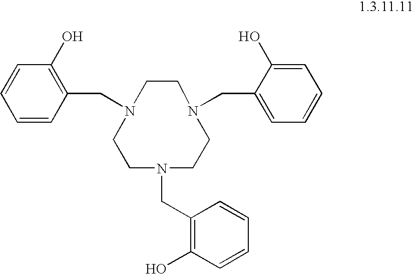

### 1.3.12 Synthesis of Polyaza Ligands with More Than One Species of Pendant Arm

### 1.3.12.1 N-(p-Toluenesulfonyl)-N′,N″-bis(diethylphosphorylmethyl)-1,4,7-triazacyclononane

From N-(p-toluenesulfonyl)-1,4,7-triazacyclononane dihydrobromide (1.3.13.31), formaldehyde and diethyl phosphite.

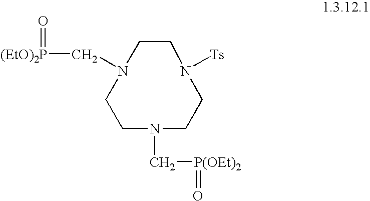

### 1.3.12.2 N,N′-Bis(diethylphosphorylmethyl)-1,4,7-triazacyclononane

From 1,4,7-triazacyclononane (1.1.3)trihydrobromide, one equivalent formaldehyde and one equivalent of diethyl phosphite. Purification of product by chromatography.

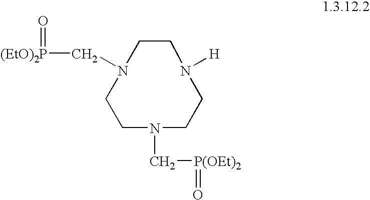

### 1.3.12.3 N,N′-Bis(dihydroxyphosphorylmethyl)-1,4,7-triazacyclononane

From 1.3.12.2 and HCl.

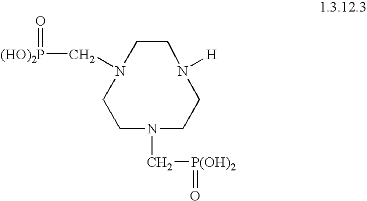

### 1.3.12.4 N-(Carboxymethyl)-N,N′-bis(dihydroxyphosphorylmethyl)-1,4,7-triazacyclononane

From 1.3.12.3, chloroacetic and NaOH,

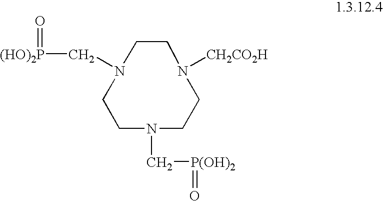

### 1.3.12.5 4-(2-Hydroxy-benzyl)-7-diethylphosphorylethyl-1,4,7,10-tetraazabicyclo[5.5.2]tetradecane

From 4-(2-hydroxybenzyl)-1,4,7,10-tetraazabicyclo[5.5.2]tetradecane (1.3.11.2), diethyl phosphite and formaldehyde solution.

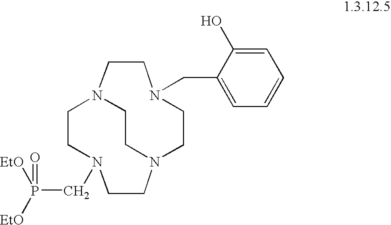

### 1.3.12.6 4-(2-hydroxy-benzyl)-7-phosphorylethyl-1,4,7,10-tetraazabicyclo [5.5.2]tetradecane

From 1.3.12.5 and HCl.

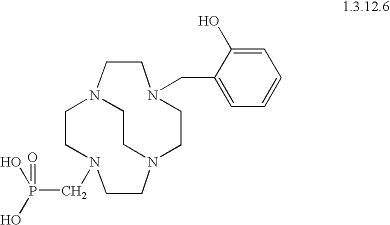

### 1.3.13 Miscellaneous Substituted Polyaza Compounds

### 1.3.13.1 1,2-Bis-(4,7-benzyloxycarbonyl-1,4,7-triazacyclononan-1-yl)ethane

From 1,2-bis-(1,4,7-triazacyclononan-1-yl)ethane (1.1.28)polyhydrobromide, potassium carbonate and benzyl chloroformate.

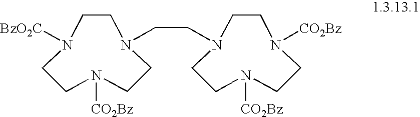

### 1.3.13.2 N-(p-Toluenesulfonyl)-N′,N″-Bis-(benzyloxycarbonyl)-1,4,7-triazacyclononane

From N-(p-toluenesulfonyl)-1,4,7-triazacyclononane dihydrobromide (1.3.13.31), K₂CO₃ and benzyl chloroformate.

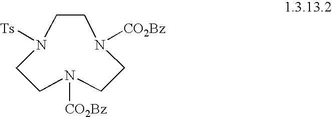

### 1.3.13.3 N-(p-Toluenesulfonyl)-N″-benzyloxycarbonyl-1,4,7-triazacyclononane

From N-(p-toluenesulfonyl)-N′,N″-bis(benzyloxycarbonyl)-1,4,7-triazacyclononane (1.3.13.2) and trimethylsilyl iodide.

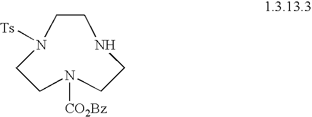

### 1.3.13.4 1,2-Bis[(1-p-toluenesulfonyl)-4-benzyloxycarbonyl-1,4,7-triazacyclonon-7-yl]ethane

From 1-(p-toluenesulfonyl)-4-benzyloxycarbonyl-1,4,7-triazacyclononane (1.3.13.3), potassium carbonate and dibromoethane.

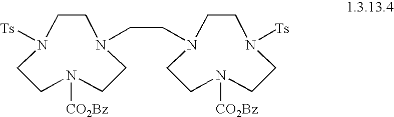

### 1.3.13.5 N,N′,N″-Tris(phenylacetyl)-1,4,7-triazacyclononane

From 1,4,7-triazacyclononane (1.1.3), diethyl phenylacetylphosphonate [PhCH₂COP(O)(OEt)₂].

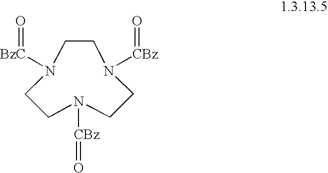

### 1.3.13.6 N,N′,N″-Tris(2,3-Epoxypropyl-1,4,7-Triazacyclononane

From 1,4,7-triazacyclononane (1.1.3) and epibromohydrin.

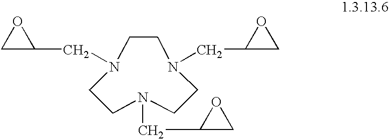

### 1.3.13.7 N,N′,N″-Tri-allyl-1,4,7-triazacyclononane

From 1,4,7-triazacyclononane (1.1.3), sodium hydride and allyl bromide.

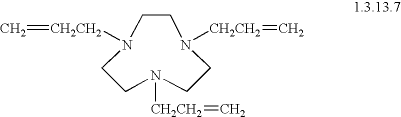

### 1.3.13.8 N,N′,N″-Tris(benzyloxycarbonyl)-1,4,7-triazacyclononane

From 1,4,7-triazacyclononane (1.1.3), benzyl chloroformate and sodium carbonate.

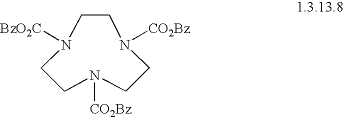

### 1.3.13.9 N,N′-Bis(benzyloxycarbonyl)-1,4,7-triazacyclononane

From N,N′,N″-tris(benzyloxycarbonyl)-1,4,7-triazacyclononane (1.3.13.8) and iodotrimethylsilane.

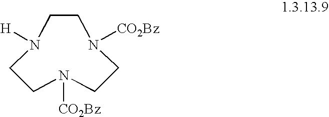

### 1.3.13.10 N,N′-Bis(benzyloxycarbonyl)-N″-(2-bromoethyl)-1,4,7-triazacyclononane

From N,N′-bis(benzyloxycarbonyl)-1,4,7-triazacyclononane (1.3.13.9), dibromoethane and potassium carbonate.

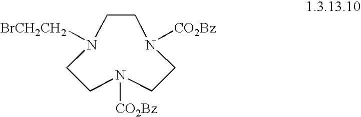

### 1.3.13.11 N-p-Toluenesulfonyl-N′,N″-ditrifluoroacetyl-1,4,7-triazacyclononane

From N-p-toluenesulfonyl-1,4,7-triazacyclononane (1.3.13.31), potassium carbonate and trifluoroacetic anhydride.

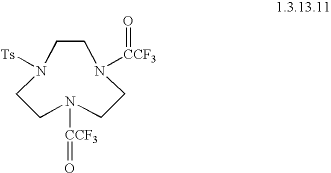

### 1.3.13.12 N-p-Toluenesulfonyl-N′-benzyl-1,4,7-triazacyclononane

From N-(p-toluenesulfonyl-1,4,7-triazacyclononane (1.3.13.31), sodium hydride and benzyl bromide.

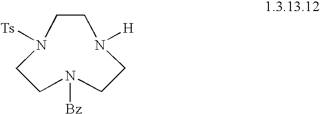

### 1.3.13.13 N-p-Toluenesulfonyl-N′,N″-dibenzyl-1,4,7-triazacyclononane

From N-(p-toluenesulfonyl-1,4,7-triazacyclononane (1.3.13.31), sodium hydride and benzyl bromide.

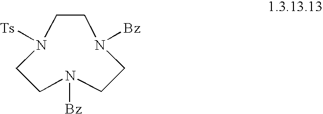

### 1.3.13.14 1,2-Bis(N-p-toluenesulfonyl-N′-benzyl)-1,4,7-triazacyclononan-1-yl) ethane

From N-p-toluenesulfonyl-N′-benzyl-1,4,7-triazacyclononane (1.3.13.12), dibromoethane and potassium carbonate.

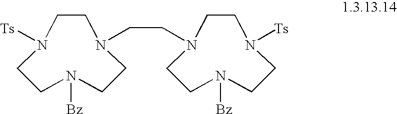

### 1.3.13.15 1,2-Bis(N,N′-ditrityl-1,4,7-triazacyclononan-1-yl)ethane

From 1,2-Bis(1,4,7-triazacyclononane)ethane (1.1.28), potassium carbonate and trityl chloride.

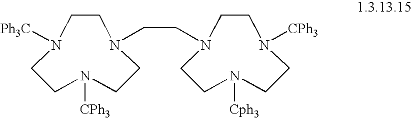

### 1.3.13.16 Spiro [4,8]-4,7-d]-p-toluenesulfonyl-4,7-diaza-1-azotridecane halide

From 1,4,7-triazacyclononane-N,N′-di-p-toluenesulfonyl hydrobromide (1.3.13.32), diiodobutane and potassium carbonate.

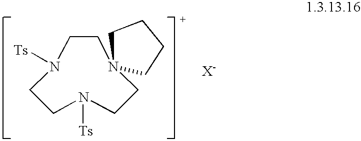

### 1.3.13.17 Tetrakis(p-toluenesulfonyl)-1,4,7,10-tetraazacyclotetradecane

From N,N′,N″-tris(p-toluenesulfonyl)diethylenetriamine (1.3.13.18), potassium carbonate and bis(2-p-toluenesulfonyloxyethyl)-N-(p-toluenesulfonyl) amine (1.3.13.19).

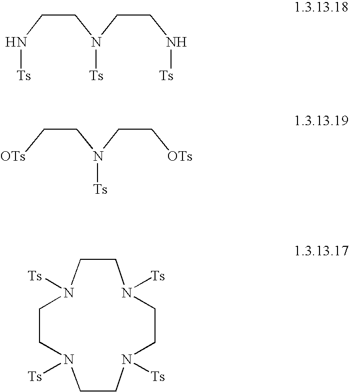

### 11.3.13.20 1,7-Bis(p-toluenesulfonyl)-4-benzyl-1,4,7-triazaheptane

From benzylamine, (2-p-toluenesulfonyoxyl)-N-(p-toluenesulfonyl)-ethylamine (1.3.13.21) and potassium carbonate.

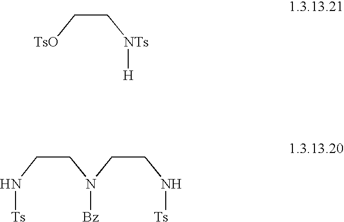

### 1.3.13.22 N-Trityldiethanolamine

From diethanolamine and trityl chloride.

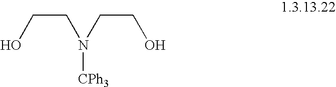

### 1.3.13.23 N-Trityl-bis(2-p-toluenesulfonyloxyethyl)amine

From N-trityldiethanolamine and p-toluenesulfonyl chloride.

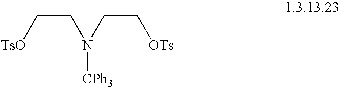

### 1.3.13.24 1,7-di-(p-toluenesulfonyl)-4-benzyl-10-trityl-1,4,7,10-tetraazacyclotetradecane

From 1,7-di-p-toluenesulfonyl-4-benzyl-1,4,7-triazaheptane (1.3.13.20), sodium hydride and N-trityl-di-p-toluenesulfonyldiethanolamine (1.3.13.23).

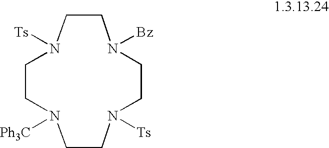

### 1.3.13.25 1,7-Di-(p-toluenesulfonyl)-1,4,7,10-tetraazacyclotetradecane

From 1,7-di-(p-toluenesulfonyl)-4-benzyl-10-trityl-1,4,7,10-tetraazacyclotetradecane (1.3.13.24) reduced by H₂ and Pd/C.

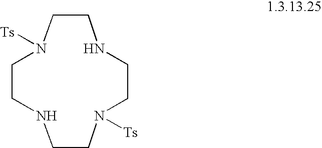

### 1.3.13.26 1,7-Di-(p-toluenesulfonyl)-4-benzyl-1,4,7,10-tetraazacyclotetradecane

From reduction of 1.3.13.24.

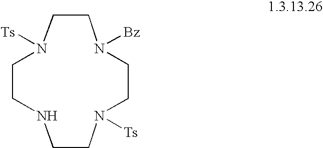

### 1.3.13.27 1,2-Bis-(4,10-di-p-toluenesulfonyl-7-benzyl-1,4,7,10-tetraazacyclotetradecan-1-yl)ethane

From 1.3.13.26 and dibromoethane.

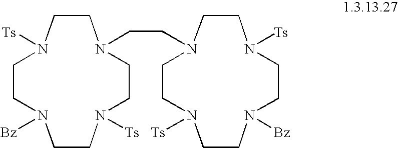

### 1.3.13.28 1,5,9,13-Tetraazatetracyclo[6,6,2,0^{1, 15}, 0^{8, 16}]hexadecane

From 1.1.6 and glyoxaldehyde.

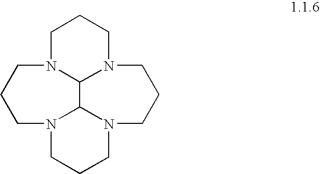

### 1.3.13.29 4,7-Diallyl-1,4,7-triazabicyclo[7,4,0]tridecane

From 1,4,7-triazabicyclo[7,4,0]tridecane trihydrobromide (1.1.14), sodium hydride and allyl bromide.

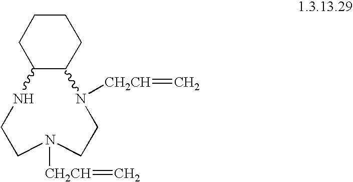

### 1.3.13.30 N-p-Toluenesulfonyl-1,4,7-triazacyclononane dihydrobromide

From N,N′,N″-Tris(p-toluenesulfonyl)-1,4,7-triazacyclononane (1.3.13.31) prepared from 1.3.13.18, dibromoethane and base) and HBr/acetic acid.

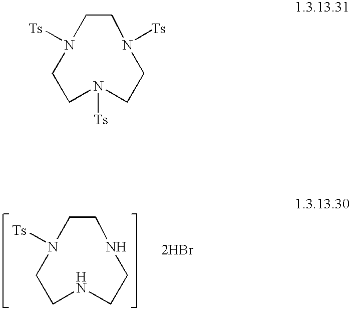

### 1.3.13.32 N,N′-Di-p-Toluenesulfonyl-1,4,7-triazacyclononane hydrobromide

a) From N,N′,N″-tris(p-toluenesulfonyl)-1,4,7-triazacyclononane (1.3.13.31 and HBr/acetic acid as the hydrobromide salt.

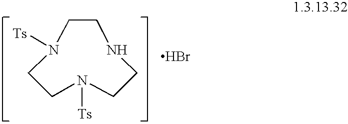

### 1.3.13.34 4,7,13-Tris(p-toluenesulfonyl)-1,4,7,10-13-pentaazabicyclo[8.5.2]heptadecane

From 1,4,7,10,13-pentaazabicyclo[8.5.2]heptadecane (1.1.26), potassium carbonate and p-toluenesulfonyl chloride.

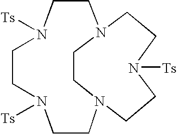

### 1.3.13.35 1,2-Bis(4-p-toluenesulfonyl-1,4,7-triazacyclonon-1-yl)ethane

From 1,2-bis(4,7-di-p-toluenesulfonyl-1,4,7-triazacyclonon-1-yl)ethane (1.1.30) and sulphuric acid.

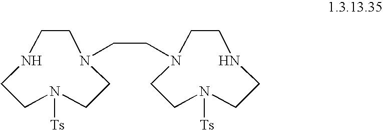

### 1.3.13.36 N,N′-(Di-p-toluenesulfonyl)-N″-benzyl-1,4,7-Triazacyclononane

a) From N,N″-(p-toluenesulfonyl)-4-benzyl diethylenetriamine (1.3.13.20), sodium hydride and ethylene glycol di-p-toluenesulfonate (1.1.12).

b) From N,N′-bis(p-toluenesulfonyl)-1,4,7-triazacyclononane (1.3.13.32), sodium hydride and benzyl bromide.

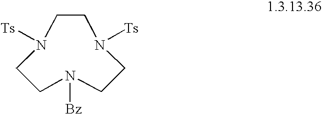

### 1.3.13.37 N-(p-Toluenesulfonyl)-N′-trityl-1,4,7-triazacyclononane

From N-(p-toluenesulfonyl-1,4,7-triazacyclononane dihydrobromide (1.3.13.30), sodium hydride and trityl chloride.

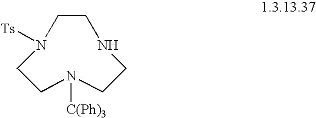

### 1.3.13.39 4,7-diallyl-1,4,7,10-tetraazabicyclo[5.5.2]tetradecane

From 1,4,7,10-tetraazabicyclo[5.5.2]tetradecane (1.1.4), allyl bromide and base.

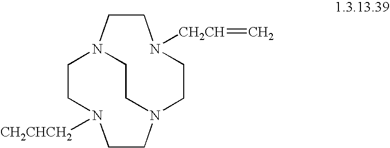

### 1.3.14 Forms of the Chelator N,N′,N″-Tris-(dihydroxyphosphorylmethyl)-1,4,7-triazacyclononane That Include Alkali and Alkaline Earth Metal Cations

### 1.3.14.1 Mono-calcium, Mono-sodium Form

To N,N′,N″-Tris-(dihydroxyphosphorylmethyl)-1,4,7-triazacyclononane was added equimolar quantities of Ca(OH)₂ and NaOH to obtain the mono-calcium, mono-sodium form of the chelator. This form is highly soluble in water and can be stored and administered in aqueous solution. Alternatively, this form can be lyophilized to a powder that can be reconstituted in aqueous solution by adding water or saline solution or the like prior to its administration.

### 1.3.14.2 Mono-magnesium, Mono-sodium Form

To N,N′,N″-Tris-(dihydroxyphosphorylmethyl)-1,4,7-triazacyclononane was added equimolar quantities of Mg(OH)₂ and NaOH to obtain the mono-magnesium, mono-sodium form of the chelator. This complex is highly soluble in water and can be stored and administered in aqueous solution. Alternatively, this form can be lyophilized to a powder that can be reconstituted in aqueous solution by adding water or saline solution or the like prior to its administration.

### 1.3.14.3 Tri-sodium Form

To N,N′,N″-Tris-(dihydroxyphosphorylmethyl)-1,4,7-triazacyclononane was added three equivalent molar quantities of NaOH to obtain the tri-sodium form of the chelator. This form is highly soluble in water and can be stored and administered in aqueous solution. Alternatively, this form can be lyophilized to a powder that can be reconstituted in aqueous solution by adding water or saline solution or the like prior to its administration.

### 1.3.14.3 1.5-Equivalent Calcium Form

To N,N′,N″-Tris-(dihydroxyphosphorylmethyl)-1,4,7-triazacyclononane was added 1.5 equivalents of Ca(OH)₂ to obtain the 1.5-equivalent calcium form of the chelator. This form is less soluble in water than the mono-calcium, mono-sodium form and can be stored and administered as an aqueous suspension. Alternatively, this form can be lyophilized to a powder that can be used to form an aqueous suspension by adding water or saline solution or the like prior to its administration.

### EXAMPLE 2

This example illustrates the relatively low toxicity of a representative example of the chelators of this invention toward nonproliferating mammalian cells in vitro.

To mature, nonreplicating cultures of HFF (human foreskin fibroblasts) kept in maintenance media was added N,N′,N″-tris(dihydroxyphosphorylmethyl)-1,4,7-triazacyclononane at a concentration of 0.3 mM. No effect on the resting cells was observed over a five-day period of observation.

### EXAMPLE 3

This example illustrates the low in vivo toxicity of a representative example of the chelators of this invention upon administration to mice.

Laboratory mice were treated by the intravenous administration of 3.0 mM/kg intravenously of the sodium salt of N,N′,N″-tris(dihydroxyphosphoryl-methyl)-1,4,7-triazacyclononane as a single intravenous dose. Over 50% of the mice thus treated survived for over 14 days following such administration, thus demonstrating that the acute LD₅₀ of this agent is in excess of 3.0 mM/kg. This in vivo LD₅₀ toxicity dose results in an instantaneous in vivo concentration which is orders of magnitude greater than the dose of this agent which inhibits mammalian cell replication in vitro (0.009 mM/L).

### EXAMPLE 4

This example demonstrates the relatively subacute toxicity of a representative example of the chelators of this invention upon administration intravenously in repeated doses to rats.

Ten male Sprague Dawley rats 29 days old and weighing between 73.4 and 87.8 grams at the beginning of the experiment were randomized, employing the block stratification method, into two groups consisting of five rats each. On each of days 1, 2, 3, 6, 7, 8, 9, 10,13 and 14 of the experiment one set of rats received an intravenous dose of N,N′,N″-tri(dihydroxyphosphoryl methyl)-1,4,7-triazacyclononane equal to 0.05 millimoles per kg of initial body weight (experimental group) while the other group received an equivalent volume of normal saline solution. The weights of the animals were recorded three times per week and the animals were sacrificed on the 28th day, major organs removed and weighed and tissues removed for microscopic examination.

There was no statistically significant difference in weight or rate of weight gain between the experimental and control group of rats, either during the period of injections or in the two-week post-injection period. There were no differences observed between the weights of major organs of the experimental vs. the control group. There were no differences between the tissues of the experimental vs. the control group upon microscopic examination of the major organs obtained at the time of necropsy.

### EXAMPLE 5

This example demonstrates the inhibition of iron-catalyzed free radical generation through Fenton Reactions (iron catalyzed Haber-Weiss pathways).

Employing published methods, as described in “Quantitative Effects of Iron Chelators on Hydroxyl Radical Production by the Superoxide-Driven Fenton Reaction,” J. B. Smith, J. C. Cusumano, C. F. Babbs, Free Rad. Res. Comms. 1990, Vol. 8, No. 2, 101-106, the ability of Fe(III) complexed to N,N′,N″-tri(dihydroxy-phosphoryl methyl)-1,4,7-triazacyclononane to support Fenton reactions was evaluated. Fe(III) complexed by ethylene diamine tetraacetic acid (EDTA) was used as a positive control and supported the Fenton reaction yielding hydroxyl radicals while Fe(III) complexed to N,N′,N″-tri(dihydroxyphosphoryl methyl)-1,4,7-triaza-cyclononane failed to show evidence of hydroxyl ion formation above background.

### EXAMPLE 6

This example demonstrates that the inhibition of bacterial replication properties of cyclic polyaza chelators with high specificity and affinity for first transition series elements is not adversely affected by the presence of increased Ca(II) concentration in the bacterial environment while the same properties of linear chelators, that do not possess such high affinity and specificity, are adversely affected. This effect is unexpected since the presence of cations other than those of first transition series elements would be expected to inhibit the rate and extent of complexation of the first transition series elements by both the cyclic and linear chelators, albeit to differing degrees.

Two chelators were compared in terms of their minimum inhibitory concentrations (MIC) against the common skin bacteria Corynebacterium Xerosis in media containing either 3.7 mg/L or 137 mg/L of Ca(II). The two chelators were N,N′,N″-tri(dihydroxyphosphoryl methyl)-1,4,7-triazacyclononane (within the scope of the present invention) and diethylene triamine pentaacetic acid (DTPA) (outside the scope of the present invention). The MIC for the triazacyclononane was 8 μg/mL in medium containing 3.7 mg/L of Ca(II), and 5 μg/mL in medium containing 137 mg/L of Ca(II). The MIC for the DTPA, by contrast, was 36 μg/mL in medium containing 3.7 mg/L of Ca(II), and 320 μg/mL in medium containing 137 mg/L of Ca(II). The chelator within the scope of the present invention is clearly superior in media containing increased Ca(II) concentration to the linear chelator in terms of its ability to inhibit bacterial replication.

### EXAMPLE 7

This example compares (a) complexes of alkaline earth metal cations such as Mg(II) and Ca(II) and cyclic polyaza chelators with high specificity and affinity for first transition series elements with (b) complexes of Na(I) (an alkali metal cation) and the same cyclic polyaza chelators in terms of the ability of each complex to mitigate ischemia and ischemia-reperfusion injury and to impart cardioprotection. In this experiment, hearts were exposed to up to 2.7 mM concentrations of these complexes prior to onset of ischemia. Unexpectedly, Ca(II) and Mg(II) complexes of the chelator, notably Ca(II) complexes, demonstrated greater efficacy in mitigating ischemia and ischemia-reperfusion injury and in affording cardioprotection than Na(I) complexes/salts of this same chelator. These findings demonstrate the unexpectedly improved biological efficacy of Ca(II) and Mg(II) complexes of cyclic polyaza chelators.

Hearts from male Wistar rats (330-370 g), anesthetized with diethyl ether inhalation, were perfused with Krebs-Henseleit buffer at 37° C. and gassed with carbogen in a working mode as described in Cardiovascular Research 30: 781-787 (1995). After an initial ten-minute aerobic, normothermic perfusion period, the hearts were subjected to 30 minutes of global, no-flow, normothermic ischemia. After ischemia, the first 5 minutes of reperfusion was performed under Langendorff perfusion in order to allow restoration of sinus rhythm. Working perfusion was then applied for an addition ten minutes to measure recovery of cardiac function. Cardiac function parameters were measured just before the induction of ischemia and at the end of 15 minutes of reperfusion. The cardiac function parameters measured were heart rate (HR), coronary artery flow (CF), aortic artery flow (AF), cardiac output (CO), left ventricular developed pressure (LVDP), positive and negative first derivatives of left ventricular pressure (+/−dP/dtₘₐₓ), left ventricular end-diastolic pressure (LVEDP), and incidence of ventricular fibrillation (VF) on reperfusion. Cardioprotective effects were considered to be achieved if increases in CF, AF, CO, LVDP and decreases in LVEDP and incidence of VF were observed following reperfusion in rats exposed to the salt/complex of the chelator.

The effects on cardiac parameters before onset of ischemia and following reperfusion were assessed employing medium plus saline and medium containing varying concentrations of the (1)trisodium, (2) monomagnesium [Mg(II)], monosodium, and (3) monocalcium [Ca(II)], monosodium complexes of the chelator N,N′,N″-tri(dihydroxyphosphoryl methyl)-1,4,7-triazacyclononane. The effects of (1) and (2) were studied at concentrations of 0.1, 0.3, 0.9, and 2.7 mM, and the effects of (3) were studied at 0.001, 0.05, 0.01, 0.05, 0.1, 0.3, 0.9, and 2.7 mM. Changes in cardiac functional parameters were considered significant if they varied from saline treated controls at the p<0.05 level. At least eight rat hearts were evaluated at each concentration for each complex and in saline treated controls.

Except for a decrease in+dP/dtₘₐₓ observed at 2.7 mM concentration of the monomagnesium monosodium scomplex, no effect of any of the three complexes of the chelator were observed in any cardiac functional parameters measured prior to onset of ischemia, demonstrating low cardiotoxicity of these complexes.

No effect of the trisodium complex (1) of the chelator was observed on any cardiac functional parameters measured after 15 minutes of reperfusion.

For the monomagnesium [Mg(II)]monosodium complex (2), increases in AF and CO and a decrease in LVEDP and a decreased incidence of VF were observed after 15 minutes of reperfusion at concentrations of 0.3, 0.9, and 2.7 mM. An increase in LVDP was observed at 0.9 mM concentration. The peak effect of the complex was deemed to have occurred at 0.9 mM concentration.

For the monocalcium [Ca(II)]monosodium salt complex (3), increases in AF, CO, and LVDP and decreases in LVEDP and incidences of VR were observed at 0.005, 0.01, 0.05, and 0.1 mM concentrations, and increases in AF and CO and a decrease in the incidence of AF was observed at 0.3 mM concentration. The peak effect was maintained throughout the effective dose range.

To summarize, after 15 minutes of ischemia followed by reperfusion, the tridosium complex produced no beneficial result, while the monomagnesium, monosodium complex and monocalcium, monosodium complex produced multiple significant beneficial results. The beneficial results obtained with the monocalcium, monosodium complex involved more functional parameters and were observed at lower concentrations and over a wider range of concentrations than were observed with the monomagnesium, monosodium complex.

The foregoing is offered primarily for purposes of illustration. Further variations, modifications, and embodiments that still fall within the spirit and scope of the invention will be readily apparent to those skilled in the art.

1. A pharmaceutical composition comprising (a) a complex of (i) a cyclic polyaza chelator having complexing affinity for first transition series elements and (ii) a cation of a member selected from the group consisting of calcium and magnesium and (b) a pharmacologically acceptable carrier.

2. The pharmaceutical composition of claim 1 in which said cyclic polyaza chelator is a chelator having the formula 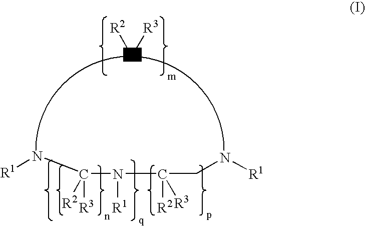
wherein:
m, n, and p are each independently 2 or 3;
q is 1 or 2;
R² and R³ are each independently selected from the group consisting of H, alkyl, alkenyl, aryl, arylalkyl, alkoxy, alkylthio, alkenoxy, alkenylthio, aryloxy, arylthio, alkyl interrupted by oxa, alkenyl interrupted by oxa, alkyl interrupted by thia, alkenyl interrupted by thia, aryloxyalkyl, alkoxyaryl, aminoalkyl, aminoalkenyl, aminoaryl, aminoarylalkyl, hydroxyalkyl, hydroxyalkenyl, hydroxyaryl, hydroxyarylalkyl, and halogen-substituted versions thereof;
R¹ is a member selected from the group consisting of R², R³ and radicals of the formula: 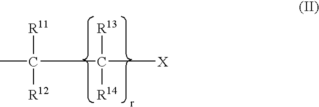
wherein:
R¹¹, R¹², and R¹³ are each independently selected from the group consisting of H, alkyl, alkenyl, aryl, arylalkyl, alkoxy, alkylthio, alkenoxy, alkenylthio, aryloxy, arylthio, alkyl interrupted by oxa, alkenyl interrupted by oxa, alkyl interrupted by thia, alkenyl interrupted by thia, aryloxyalkyl, alkoxyaryl, aminoalkyl, aminoalkenyl, aminoaryl, aminoarylalkyl, hydroxyalkyl, hydroxyalkenyl, hydroxyaryl, hydroxyarylalkyl, and halogen-substituted versions thereof;
R¹⁴ is a member selected from the group consisting of H, hydroxy, amino, alkyl, alkyl interrupted by oxa, alkoxy, aryl, aryloxyalkyl, alkoxyaryl, alkoxyaryl, and halogen-substituted versions thereof;
r is zero or 1; and
X is a member selected from the group consisting of alkyl, alkenyl, aryl, arylalkyl, alkoxy, alkylthio, alkenoxy, alkenylthio, aryloxy, arylthio, alkyl interrupted by oxa, alkenyl interrupted by oxa, alkyl interrupted by thia, alkenyl interrupted by thia, aryloxyalkyl, alkoxyaryl, aminoalkyl, aminoalkenyl, aminoaryl, aminoarylalkyl, hydroxyalkyl, hydroxyalkenyl, hydroxyaryl, hydroxyarylalkyl, halogen-substituted versions thereof, and radicals selected form the group consisting of: 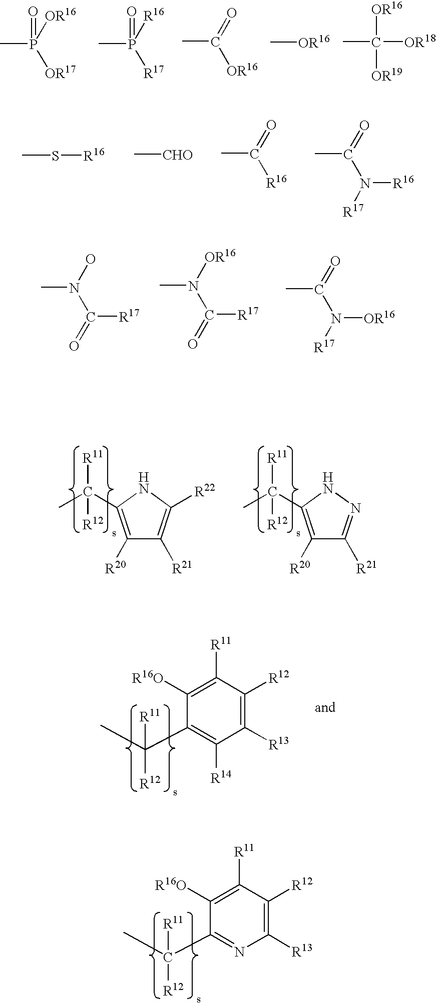
wherein,
R¹¹, R¹², R¹³ and R¹⁴ are each independently as defined above;
R¹⁶ and R¹⁷ are each independently selected from the group consisting of H, alkyl and aryl, or taken together form a ring structure;
R¹⁸ and R¹⁹ are each independently selected from the group consisting of H, alkyl, aryl, alkoxy, alkyl interrupted by oxa, aryloxyalkyl, alkoxyaryl, and halogen-substituted versions thereof;
R²⁰, R²¹ and R²² are each independently selected from the group consisting of H, alkyl, alkenyl, aryl, arylalkyl, alkoxy, alkylthio, alkenyloxy, alikenylthio, aryloxy, aminoalkyl, aminoalkenyl, aminoaryl, aminoarylakyl, hydroxyalkyl, hydroxyalkenyl, hydroxyaryl, and hydroxyarylalkyl; and
s is an integer of from 1 to 3,
and wherein, optionally, any two of R¹, R², and R³ are combined to form a ring structure;
and dimers of Formula I, said dimers being formed by the covalent attachment of two complexing agents of Formula I through a linking group having from 1 to 6 carbon atoms; and physiological salts thereof.

3. The pharmaceutical composition of claim 2 wherein m, n, and p are each 2.

4. The pharmaceutical composition of claim 2 wherein q is 1.

5. The pharmaceutical composition of claim 2 wherein said cation is calcium.

6. The pharmaceutical composition of claim 2 wherein m, n, and p are each 2, q is 1, and said cation is calcium.

7. The pharmaceutical composition of claim 2 wherein all alkyl are C₁-C₄ alkyl.

8. The pharmaceutical composition of claim 2 wherein all alkyl are C₁-C₄ alkyl, all alkenyl are vinyl, all aryl are phenyl, all aralkyl are phenethyl or benzyl, all cycloalkyl are cyclopentyl or cyclohexyl, and all halogens are chlorine or fluorine.

9. The pharmaceutical composition of claim 2 wherein R² and R³ are each independently selected from the group consisting of H, alkyl, alkenyl, aryl, and aralkyl.

10. The pharmaceutical composition of claim 2 wherein R² and R³ are each independently selected from the group consisting of H and C₁-C₄ alkyl.

11. The pharmaceutical composition of claim 2 wherein R² and R³ are each H.

12. The pharmaceutical composition of claim 2 wherein R² and R³ are each H and q is 1.

13. The pharmaceutical composition of claim 2 wherein R¹ is

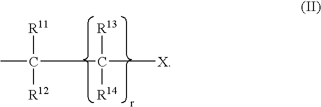

14. The pharmaceutical composition of claim 2 wherein q is 1, said cation is calcium, and R¹ is

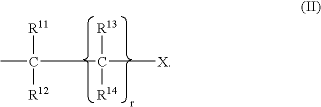

15. The pharmaceutical composition of claim 14 wherein X is a member selected from the group consisting of alkyl, alkenyl, aryl, arylalkyl, and radicals selected from the group consisting of:

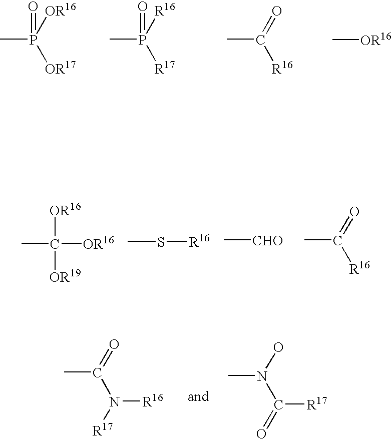

16. The pharmaceutical composition of claim 15 wherein R¹⁶, R¹⁷, R¹⁸, and R¹⁹ are independently selected from the group consisting of H and C₁-C₄ alkyl.

17. The pharmaceutical composition of claim 14 wherein X is a member selected from the group consisting of alkyl, alkenyl, aryl, arylalkyl, and radicals selected from the group consisting of:

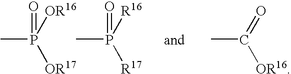

18. The pharmaceutical composition of claim 17 wherein R¹⁶ and R¹⁷ are independently selected from the group consisting of H and C₁-C₄ alkyl.

19. The pharmaceutical composition of claim 14 wherein X is a member selected from the group consisting of:

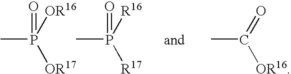

20. The pharmaceutical composition of claim 19 wherein R¹⁶ and R¹⁷ are independently selected from the group consisting of H and C₁-C₄ alkyl.

21. The pharmaceutical composition of claim 2 wherein R² and R³ are each independently selected from the group consisting of H, alkyl, alkenyl, aryl, and aralkyl, and R¹ is a member selected from the group consisting of H, alkyl, alkenyl, aryl, aralkyl, and 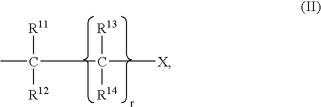
in which R¹¹, R¹², and R¹³ are each independently selected from the group consisting of H, alkyl, alkenyl, aryl, and arylalkyl, and R¹⁴ is a member selected from the group consisting of H, hydroxy, amino, and alkyl.

22. The pharmaceutical composition of claim 2 wherein R¹ is 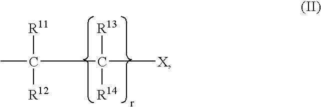
in which R¹¹, R¹², and R¹³ are each independently selected from the group consisting of H, alkyl, alkenyl, aryl, and arylalkyl, and R¹⁴ is a member selected from the group consisting of H, hydroxy, amino, and alkyl.

23. The pharmaceutical composition of claim 2 wherein:
R¹ is 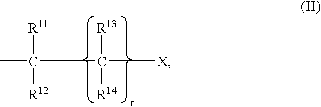
in which R¹¹, R¹², and R¹³ are each independently selected from the group consisting of H and C₁-C₄ alkyl, R¹⁴ is a member selected from the group consisting of H and C₁-C₄ alkyl, and X is a member selected from the group consisting of 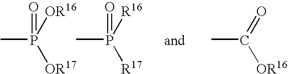
in which R¹⁶ and R¹⁷ are each independently H or C₁-C₄ alkyl;
R² and R³ are each independently selected from the group consisting of H and C₁-C₄ alkyl;
m, n, and p are each 2;
q is 1; and
said cation is calcium.

24. The pharmaceutical composition of claim 2 wherein R¹ is 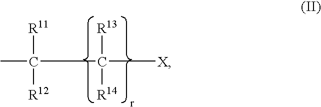
in which R¹¹, R¹², and R¹³ are each independently selected from the group consisting of H and C₁-C₄ alkyl, and R¹⁴ is a member selected from the group consisting of H and C₁-C₄ alkyl.

25. The pharmaceutical composition of claim 2 wherein R¹ is dihydroxyphosphorylmethyl, R² is H, R³ is H, m is 2, n is 2, p is 2, and q is 1.

26. The pharmaceutical composition of claim 25 in which said cation is calcium.

27. A method for enhancing the biological activity of a cyclic polyaza chelator having complexing affinity for first transition series elements, said method comprising administering said chelator as a complex with a cation selected from the group consisting of calcium and magnesium.

28. The method of claim 27 in which said cation is calcium.

29. A method for providing neuroprotection or cardioprotection in a patient, said method comprising administering to said patient an effective amount of a pharmaceutical composition of claim 1.

30. A method for mitigating damage to the central nervous system of a patient suffering from ischemic stroke, seizure or trauma, said method comprising administering to said patient an effective amount of a pharmaceutical composition of claim 1.

31. A method for mitigating damage to the heart of a patient suffering a heart attack or arrhythmia, said method comprising administering to said patient an effective amount of a pharmaceutical composition of claim 1.

32. A method for mitigating ischemia or ischemia-reperfusion injury in a patient, said method comprising administering to said patient an effective amount of a pharmaceutical composition of claim 1.

33. A method for mitigating ischemia or ischemia-reperfusion injury in a patient that has undergone cardiopulmonary bypass, said method comprising administering to said patient an effective amount of a pharmaceutical composition of claim 1.

34. A method for mitigating ischemia or ischemia-reperfusion injury in a patient that has undergone vascular surgery, said method comprising administering to said patient an effective amount of a pharmaceutical composition of claim 1.

35. A method for mitigating ischemia or ischemia-reperfusion injury in transplanted tissue in a patient that has undergone tissue transplant, said method comprising administering to said patient an effective amount of a pharmaceutical composition of claim 1.

36. A method for providing neuroprotection or cardioprotection in a patient, said method comprising administering to said patient an effective amount of a pharmaceutical composition of claim 2.

37. A method for enhancing the biological activity of a cyclic polyaza chelator having complexing affinity for first transition series elements, said method comprising administering said chelator as a pharmaceutical composition of claim 2.

38. A method for mitigating ischemia or ischemia-reperfusion injury in a patient, said method comprising administering to said patient an effective amount of a pharmaceutical composition of claim 2.

39. A method for mitigating damage to the central nervous system of a patient suffering from ischemic stroke, seizure or trauma, said method comprising administering to said patient an effective amount of a pharmaceutical composition of claim 2.

40. A method for mitigating damage to the heart of a patient suffering a heart attack or arrhythmia, said method comprising administering to said patient an effective amount of a pharmaceutical composition of claim 2.

41. A method for enhancing the biological activity of a cyclic polyaza chelator having complexing affinity for first transition series elements, said method comprising administering said chelator as a pharmaceutical composition of claim 23.

42. A method for mitigating ischemia or ischemia-reperfusion injury in a patient, said method comprising administering to said patient an effective amount of a pharmaceutical composition of claim 23.

43. A method for providing neuroprotection or cardioprotection in a patient, said method comprising administering to said patient an effective amount of a pharmaceutical composition of claim 23.

44. A method for mitigating damage to the central nervous system of a patient suffering from ischemic stroke, seizure or trauma, said method comprising administering to said patient an effective amount of a pharmaceutical composition of claim 23.

45. A method for mitigating damage to the heart of a patient suffering a heart attack or arrhythmia, said method comprising administering to said patient an effective amount of a pharmaceutical composition of claim 23.

46. A method for enhancing the biological activity of a cyclic polyaza chelator having complexing affinity for first transition series elements, said method comprising administering said chelator as a pharmaceutical composition of claim 25.

47. A method for mitigating ischemia or ischemia-reperfusion injury in a patient, said method comprising administering to said patient an effective amount of a pharmaceutical composition of claim 25.

48. A method for providing neuroprotection or cardioprotection in a patient, said method comprising administering to said patient an effective amount of a pharmaceutical composition of claim 25.

49. A method for mitigating damage to the central nervous system of a patient suffering from ischemic stroke, seizure or trauma, said method comprising administering to said patient an effective amount of a pharmaceutical composition of claim 25.

50. A method for mitigating damage to the heart of a patient suffering a heart attack or arrhythmia, said method comprising administering to said patient an effective amount of a pharmaceutical composition of claim 25.
